(19) United States
(12) Patent Application Publication
(10) Pub. No.: US 2024/0327831 A1
(43) Pub. Date: Oct. 3, 2024

(54) COMPOSITIONS COMPRISING EXON SKIPPING OLIGONUCLEOTIDE CONJUGATES FOR TREATING MUSCULAR DYSTROPHY

(71) Applicant: Sarepta Therapeutics, Inc., Cambridge, MA (US)

(72) Inventors: Arani CHANDA, Cambridge, MA (US); Willow DILUZIO, Cambridge, MA (US); Pooja SANE, Cambridge, MA (US); Shivanga NAIK, Cambridge, MA (US)

(73) Assignee: Sarepta Therapeutics, Inc., Cambridge, MA (US)

(21) Appl. No.: 18/257,547

(22) PCT Filed: Dec. 22, 2021
(86) PCT No.: PCT/US2021/064848

§ 371 Date: Jun. 14, 2023

Related U.S. Application Data
(60) Provisional application No. 63/129,752, filed on Dec. 23, 2020.

(51) Int. Cl. C12N 15/113; A61K 9/19; A61K 47/26; A61K 47/64

(52) U.S. Cl. CPC C12N 15/113; A61K 9/19; A61K 47/26; A61K 47/645; C12N 2310/11; C12N 2310/3513; C12N 2320/33

(57) ABSTRACT

Composition comprising: (a) an antisense oligonucleotide conjugate comprising a cell penetrating peptide covalently attached to a nucleic acid analog, or a pharmaceutically acceptable salt thereof, wherein the cell penetrating peptide includes at least two positively charged amino acids; (b) one or more surfactants; (c) one or more sugars; and (d) one or more buffering agents are provided herein.

### RELATED APPLICATION

This patent application is a 371 application of PCT/US2021/064848, filed Dec. 22, 2021, which claims the benefit of U.S. Provisional Application Ser. No. 63/129,752, filed Dec. 23, 2020. The entire teachings of the above-referenced applications are incorporated by reference in their entireties.

### SEQUENCE LISTING

The instant application contains a Sequence Listing which has been submitted electronically in ASCII format and is hereby incorporated by reference in its entirety. Said ASCII copy, created on Dec. 21, 2021, is named 8169_51_WO00_SL.txt and is 16,618 bytes in size.

### FIELD OF THE DISCLOSURE

The present disclosure relates to compositions comprising antisense oligonucleotides, or pharmaceutically acceptable salts thereof, suitable for exon skipping in the human dystrophin gene.

### BACKGROUND OF THE DISCLOSURE

Duchenne muscular dystrophy (DMD) is caused by a defect in the expression of the protein dystrophin. The gene encoding the protein contains 79 exons spread out over more than 2 million nucleotides of DNA. Any exonic mutation that changes the reading frame of the exon, or introduces a stop codon, or is characterized by removal of an entire out of frame exon or exons, or duplications of one or more exons, has the potential to disrupt production of functional dystrophin, resulting in DMD.

A less severe form of muscular dystrophy, Becker muscular dystrophy (BMD) has been found to arise where a mutation, typically a deletion of one or more exons, results in a correct reading frame along the entire dystrophin transcript, such that translation of mRNA into protein is not prematurely terminated. If the joining of the upstream and downstream exons in the processing of a mutated dystrophin pre-mRNA maintains the correct reading frame of the gene, the result is an mRNA coding for a protein with a short internal deletion that retains some activity, resulting in a Becker phenotype.

There is a need for pharmaceutical compositions comprising antisense oligonucleotides conjugates suitable for exon skipping that are useful for therapeutic methods for producing dystrophin and treating DMD.

### SUMMARY OF THE DISCLOSURE

A composition, comprising: (a) an antisense oligonucleotide conjugate comprising a cell penetrating peptide covalently attached to a nucleic acid analog, or a pharmaceutically acceptable salt thereof, wherein the cell penetrating peptide includes at least two positively charged amino acids; (b) one or more surfactants; (c) one or more sugars; and (d) one or more buffering agents.

In some aspects, the composition comprises about 50 mg to about 500 mg of the antisense oligonucleotide conjugate, or a pharmaceutically acceptable salt thereof. In some aspects, the antisense oligonucleotide conjugate in the composition is capable of binding a selected target to induce exon skipping in the human dystrophin gene.

In some aspects, the antisense oligonucleotide conjugate in the composition comprises a cell penetrating peptide that is an arginine-rich peptide. In some aspects, the antisense oligonucleotide conjugate in the composition comprises a cell penetrating peptide that is an arginine-rich peptide selected from the group consisting of —(RXR)₄—R^{a} (SEQ ID NO: 54), R-(FFR)₃-R^{a} (SEQ ID NO: 55), —B—X—(RXR)₄—R^{a} (SEQ ID NO: 56), —B—X—R-(FFR)₃-R^{a} (SEQ ID NO: 57), -GLY-R-(FFR)₃-R^{a} (SEQ ID NO: 58), -GLY-R₅—R^{a} (SEQ ID NO: 59), —R₅—R^{a} (SEQ ID NO: 60), -GLY-R₆—R^{a} (SEQ ID NO: 52) and —R₆—R^{a} (SEQ ID NO: 53), wherein R^{a} is selected from H, acyl, benzoyl, and stearoyl, and wherein R is arginine, X is 6-aminohexanoic acid, B is β-alanine, F is phenylalanine and GLY (or G) is glycine. In some aspects, the arginine-rich peptide is -GLY-R₅—R^{a} (SEQ ID NO: 59), —R₅—R^{a} (SEQ ID NO: 60), -GLY-R₆—R^{a} (SEQ ID NO: 52) or —R₆—R^{a} (SEQ ID NO: 53), wherein R is arginine and R^{a} is hydrogen or an acyl group.

In some aspects, the antisense oligonucleotide conjugate in the composition comprises a sequence that is complementary to 15 to 35 nucleobases of an exon 44, exon 45, exon 50, exon 51, exon 52, or exon 53 target region of the dystrophin pre-mRNA. In some aspects, the antisense oligonucleotide conjugate in the composition is of Formula (I):

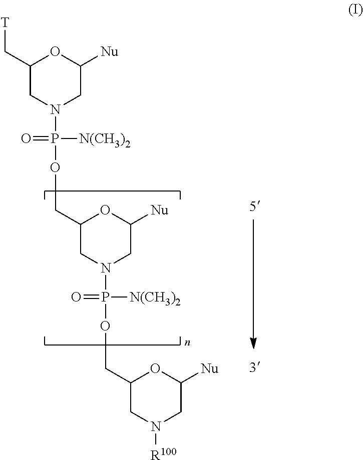

or a pharmaceutically acceptable salt thereof, in the composition wherein:
each Nu is a nucleobase which taken together form a targeting sequence;
T is a moiety selected from:

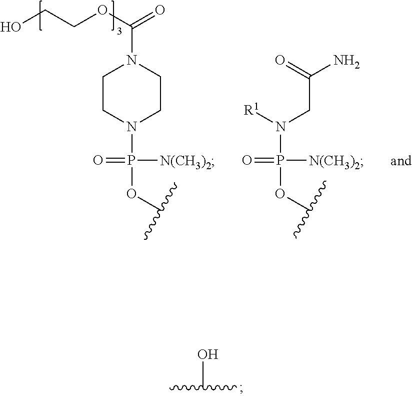

R¹⁰⁰ is the cell penetrating peptide;
each Nu from 1 to m and 5′ to 3′ corresponds to the nucleobases in one of the following:

| | | SEQ ID |
|---|---|---|
| Annealing Site | Base Sequence [5′ to 3′] | NO. |
| H51A(+66+95) | CTC CAA CAT CAA GGA AGA TGG CAT TTC TAG | 1 |
| H51A(+74+97) | ACC TCC AAC ATC AAG GAA GAT GGC | 2 |
| H51A(+70+99) | GTA CCT CCA ACA TCA AGG AAG ATG GCA TTT | 3 |
| H51A(+72+99) | GTA CCT CCA ACA TCA AGG AAG ATG GCA T | 4 |
| H51A(+68+87) | TCA AGG AAG ATG GCA TTT CT | 5 |
| H51A(+68+87) | UCA AGG AmAGm AmUGm GmCA UUU CU | 6 |
| H53A(+36+60) | GTT GCC TCC GGT TCT GAA GGT GTT C | 7 |
| H53A(+36+60) | GTT G5mC5mC T5mC5mC GGT T5mC T GAA GGT GTT 5mC | 8 |
| H53A(+36+56) | CCT CCG GTT CTG AAG GTG TTC | 9 |
| H53A(+23+47) | CTG AAG GTG TTC TTG TAC TTC ATC C | 10 |
| H53A(+32+56) | CCT CCG GTT CTG AAG GTG TTC TTG T | 11 |
| H53A(+33+60) | GTT GCC TCC GGT TCT GAA GGT GTT CTT G | 12 |
| H53A(+30+59) | TTG CCT CCG GTT CTG AAG GTG TTC TTG TAC | 13 |
| H53A(+39+62) | CTG TTG CCT CCG GTT CTG AAG GTG | 14 |
| H53A(+36+69) | CAT TCA ACT GTT GCC TCC GGT TCT GAA GGT G | 15 |
| H53A(+45+62) | CTG TTG CCT CCG GTT CTG | 16 |
| H45A(−03+19) | CAA TGC CAT CCT GGA GTT CCT G | 17 |
| H45A(−09+25) | GCT GCC CAA TGC CAT CCT GGA GTT CCT GTA AGA T | 18 |
| H45A(−03+25) | GCT GCC CAA TGC CAT CCT GGA GTT CCT G | 19 |
| H45A(−06+25) | GCT GCC CAA TGC CAT CCT GGA GTT CCT GTA A | 20 |
| H45A(−12+19) | CAA TGC CAT CCT GGA GTT CCT GTA AGA TAC C | 21 |
| H45A(−09+19) | CAA TGC CAT CCT GGA GTT CCT GTA AGA T | 22 |
| H45A(−12+16) | TGC CAT CCT GGA GTT CCT GTA AGA TAC C | 23 |
| H45A(−14+25) | GCT GCC CAA TGC CAT CCT GGA GTT CCT GTA AGA TAC | 24 |
| | CAA | |
| H45A(−08+19) | CAA TGC CAT CCT GGA GTT CCT GTA AGA | 25 |
| HM45A(−07+25) | GCT GCC CAA TGC CAT CCT GGA GTT CCT GTA AG | 26 |
| H45A(−12+22) | GCC CAA TGC CAT CCT GGA GTT CCT GTA AGA TAC C | 27 |
| H45A(−09+22) | GCC CAA TGC CAT CCT GGA GTT CCT GTA AGA T | 28 |
| H45A(−09+30) | TTG CCG CTG CCC AAT GCC ATC CTG GAG TTC CTG TAA | 29 |
| | GAT | |
| H45A(−06+22) | GCC CAA TGC CAT CCT GGA GTT CCT GTA A | 30 |
| H45A(−06+28) | GCC GCT GCC CAA TGC CAT CCT GGA GTT CCT GTA A | 31 |
| H45A(−03+22) | GCC CAA TGC CAT CCT GGA GTT CCT G | 32 |
| H45A(−03+28) | GCC GCT GCC CAA TGC CAT CCT GGA GTT CCT G | 33 |
| H45A(+9+26) | m5C-G-m5C-T-G-C-m5C-m5C-A-A-T-G-m5C-m5C-A-U-m5C- | 34 |
| | m5C | |
| H44A(−10+15) | GAT CTG TCA AAT CGC CTG CAG GTA A | 35 |
| H44A(−07+15) | GAT CTG TCA AAT CGC CTG CAG G | 36 |
| H44M(−07+17) | CAG ATC TGT CAA ATC GCC TGC AGG | 37 |
| H44A(−08+15) | GAT CTG TCA AAT CGC CTG CAG GT | 38 |
| H44A(−06+15) | GAT CTG TCA AAT CGC CTG CAG | 39 |
| H44A(−08+17) | CAG ATC TGT CAA ATC GCC TGC AGG T | 40 |
| H44A(−06+17) | CAG ATC TGT CAA ATC GCC TGC AG | 41 |
| H50D(+04−18) | GGG ATC CAG TAT ACT TAC AGG C | 42 |
| H50D(+07−18) | GGG ATC CAG TAT ACT TAC AGG CTC C | 43 |
| H50D(+07−16) | GAT CCA GTA TAC TTA CAG GCT CC | 44 |
| H50D(+07−17) | GGA TCC AGT ATA CTT ACA GGC TCC | 45 |
| H50A(−19+07) | ACT TCC TCT TTA ACA GAA AAG CAT AC | 46 |
| H50D(+07−15) | ATC CAG TAT ACT TAC AGG CTC C | 47 |
| H50A(−02+23) | GAG CTC AGA TCT TCT AAC TTC CTC T | 48 |
| H50D(+06−18) | GGG ATC CAG TAT ACT TAC AGG CTC | 49 |
| H50D(+07−20) | ATG GGA TCC AGT ATA CTT ACA GGC TCC | 50 |
| H52A(−01+24) | CTG TTC CAA ATC CTG CAT TGT TGC C | 51 |

wherein A is

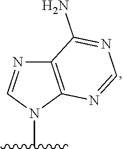 [SMILES: *n1cnc2c(N)ncnc21]

### C is

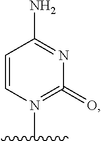

### G is

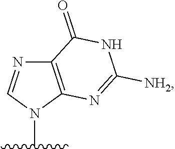 [SMILES: *n1cnc2c(=O)[nH]c(N)nc21]

### T is

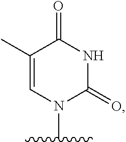 [SMILES: *n1cc(C)c(=O)[nH]c1=O]

### U is

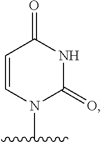 [SMILES: *n1ccc(=O)[nH]c1=O]

Gm is methylated guanine, Am is methylated adenine, and m5C is

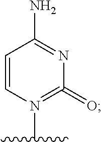

In some aspects, each Nu from 1 to n and 5′ to 3′ of the antisense oligonucleotide conjugate of Formula (I), or pharmaceutically acceptable salt thereof, in the composition corresponds to SEQ ID NO: 1, SEQ ID NO: 7, SEQ ID NO: 17, or SEQ ID NO: 51. In some aspects, each Nu from 1 to n and 5′ to 3′ of the antisense oligonucleotide of Formula (I), or pharmaceutically acceptable salt thereof, in the composition corresponds to SEQ ID NO: 1. In some aspects, each Nu from 1 to n and 5′ to 3′ of the antisense oligonucleotide of Formula (I), or pharmaceutically acceptable salt thereof, in the composition corresponds to SEQ ID NO: 7. In some aspects, each Nu from 1 to n and 5′ to 3′ of the antisense oligonucleotide of Formula (I), or pharmaceutically acceptable salt thereof, in the composition corresponds to SEQ ID NO: 17. In some aspects, each Nu from 1 to n and 5′ to 3′ of the antisense oligonucleotide of Formula (I), or pharmaceutically acceptable salt thereof, in the composition corresponds to SEQ ID NO: 51.

In some aspects, T′ in the antisense oligonucleotide conjugate of Formula (I) in the composition is

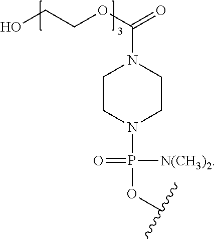

In some aspects, the antisense oligonucleotide conjugate in the composition is of Formula (II):

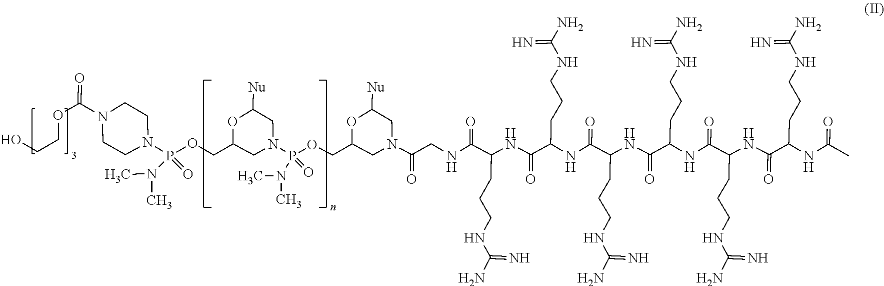

or a pharmaceutically acceptable salt thereof, each Nu from 1 to (n+1) and 5′ to 3′ corresponds to the nucleobases in one of the following:

| | SEQ |
|---|---|
| | ID |
| Base Sequence [5′ to 3′] | NO. |
| CTC CAA CAT CAA GGA AGA TGG CAT TTC TAG | 1 |
| ACC TCC AAC ATC AAG GAA GAT GGC | 2 |
| GTA CCT CCA ACA TCA AGG AAG ATG GCA TTT | 3 |
| GTA CCT CCA ACA TCA AGG AAG ATG GCA T | 4 |
| TCA AGG AAG ATG GCA TTT CT | 5 |
| UCA AGG AmAGm AmUGm GmCA UUU CU | 6 |
| GTT GCC TCC GGT TCT GAA GGT GTT C | 7 |
| GTT G5mC5mC T5mC5mC GGT T5mC T GAA GGT GTT | 8 |
| 5mC | |
| CCT CCG GTT CTG AAG GTG TTC | 9 |
| CTG AAG GTG TTC TTG TAC TTC ATC C | 10 |
| CCT CCG GTT CTG AAG GTG TTC TTG T | 11 |
| GTT GCC TCC GGT TCT GAA GGT GTT CTT G | 12 |
| TTG CCT CCG GTT CTG AAG GTG TTC TTG TAC | 13 |
| CTG TTG CCT CCG GTT CTG AAG GTG | 14 |
| CAT TCA ACT GTT GCC TCC GGT TCT GAA GGT G | 15 |
| CTG TTG CCT CCG GTT CTG | 16 |
| CAA TGC CAT CCT GGA GTT CCT G | 17 |
| GCT GCC CAA TGC CAT CCT GGA GTT CCT GTA AGA | 18 |
| T | |
| GCT GCC CAA TGC CAT CCT GGA GTT CCT G | 19 |
| GCT GCC CAA TGC CAT CCT GGA GTT CCT GTA A | 20 |
| CAA TGC CAT CCT GGA GTT CCT GTA AGA TAC C | 21 |
| CAA TGC CAT CCT GGA GTT CCT GTA AGA T | 22 |
| TGC CAT CCT GGA GTT CCT GTA AGA TAC C | 23 |
| GCT GCC CAA TGC CAT CCT GGA GTT CCT GTA AGA | 24 |
| TAC CAA | |
| CAA TGC CAT CCT GGA GTT CCT GTA AGA | 25 |
| GCT GCC CAA TGC CAT CCT GGA GTT CCT GTA AG | 26 |
| GCC CAA TGC CAT CCT GGA GTT CCT GTA AGA TAC | 27 |
| C | |
| GCC CAA TGC CAT CCT GGA GTT CCT GTA AGA T | 28 |
| TTG CCG CTG CCC AAT GCC ATC CTG GAG TTC CTG | 29 |
| TAA GAT | |
| GCC CAA TGC CAT CCT GGA GTT CCT GTA A | 30 |
| GCC GCT GCC CAA TGC CAT CCT GGA GTT CCT GTA A | 31 |
| GCC CAA TGC CAT CCT GGA GTT CCT G | 32 |
| GCC GCT GCC CAA TGC CAT CCT GGA GTT CCT G | 33 |
| m5C-G-m5C-T-G-C-m5C-m5C-A-A-T-G-m5C-m5C-A-U- | 34 |
| m5C-m5C | |
| GAT CTG TCA AAT CGC CTG CAG GTA A | 35 |
| GAT CTG TCA AAT CGC CTG CAG G | 36 |
| CAG ATC TGT CAA ATC GCC TGC AGG | 37 |
| GAT CTG TCA AAT CGC CTG CAG GT | 38 |
| GAT CTG TCA AAT CGC CTG CAG | 39 |
| CAG ATC TGT CAA ATC GCC TGC AGG T | 40 |
| CAG ATC TGT CAA ATC GCC TGC AG | 41 |
| GGG ATC CAG TAT ACT TAC AGG C | 42 |
| GGG ATC CAG TAT ACT TAC AGG CTC C | 43 |
| GAT CCA GTA TAC TTA CAG GCT CC | 44 |
| GGA TCC AGT ATA CTT ACA GGC TCC | 45 |
| ACT TCC TCT TTA ACA GAA AAG CAT AC | 46 |
| ATC CAG TAT ACT TAC AGG CTC C | 47 |
| GAG CTC AGA TCT TCT AAC TTC CTC T | 48 |
| GGG ATC CAG TAT ACT TAC AGG CTC | 49 |
| ATG GGA TCC AGT ATA CTT ACA GGC TCC | 50 |
| CTG TTC CAA ATC CTG CAT TGT TGC C | 51 |

wherein A is

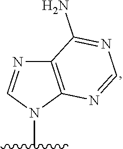 [SMILES: *n1cnc2c(N)ncnc21]

### C is

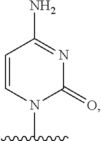 [SMILES: *n1ccc(N)nc1=O]

### G is

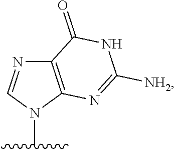 [SMILES: *n1cnc2c(=O)[nH]c(N)nc21]

### T is

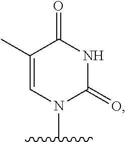 [SMILES: *n1cc(C)c(=O)[nH]c1=O]

### U is

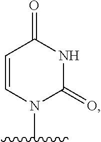 [SMILES: *n1ccc(=O)[nH]c1=O]

Gm is methylated guanine, Am is methylated adenine, and m5C is

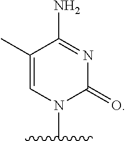 [SMILES: *n1cc(C)c(N)nc1=O]

In some aspects, each Nu from 1 to n and 5′ to 3′ of the antisense oligonucleotide conjugate of Formula (II), or pharmaceutically acceptable salt thereof, in the composition corresponds to SEQ ID NO: 1, SEQ ID NO: 7, SEQ ID NO: 17, or SEQ ID NO: 51. In some aspects, each Nu from 1 to n and 5′ to 3′ of the antisense oligonucleotide of Formula (II), or pharmaceutically acceptable salt thereof, in the composition corresponds to SEQ ID NO: 1. In some aspects, each Nu from 1 to n and 5′ to 3′ of the antisense oligonucleotide of Formula (II), or pharmaceutically acceptable salt thereof, in the composition corresponds to SEQ ID NO: 7. In some aspects, each Nu from 1 to n and 5′ to 3′ of the antisense oligonucleotide of Formula (II), or pharmaceutically acceptable salt thereof, in the composition corresponds to SEQ ID NO: 17. In some aspects, each Nu from 1 to n and 5′ to 3′ of the antisense oligonucleotide of Formula (II), or pharmaceutically acceptable salt thereof, in the composition corresponds to SEQ ID NO: 51.

In some aspects, the antisense oligonucleotide conjugate in the composition is of Formula (III):

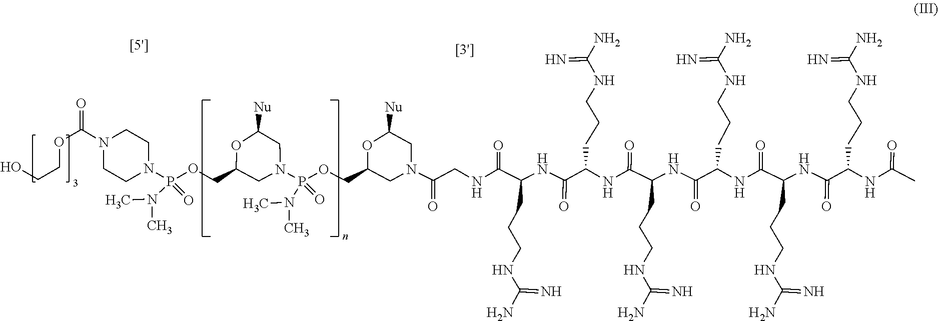

or a pharmaceutically acceptable salt thereof, each Nu from 1 to (n+1) and 5′ to 3′ corresponds to the nucleobases in one of the following:

| | SEQ |
|---|---|
| | ID |
| Base Sequence [5′ to 3′] | NO. |
| CTC CAA CAT CAA GGA AGA TGG CAT TTC TAG | 1 |
| ACC TCC AAC ATC AAG GAA GAT GGC | 2 |
| GTA CCT CCA ACA TCA AGG AAG ATG GCA TTT | 3 |
| GTA CCT CCA ACA TCA AGG AAG ATG GCA T | 4 |
| TCA AGG AAG ATG GCA TTT CT | 5 |
| UCA AGG AmAGm AmUGm GmCA UUU CU | 6 |
| GTT GCC TCC GGT TCT GAA GGT GTT C | 7 |
| GTT G5mC5mC T5mC5mC GGT T5mC T GAA GGT GTT | 8 |
| 5mC | |
| CCT CCG GTT CTG AAG GTG TTC | 9 |
| CTG AAG GTG TTC TTG TAC TTC ATC C | 10 |
| CCT CCG GTT CTG AAG GTG TTC TTG T | 11 |
| GTT GCC TCC GGT TCT GAA GGT GTT CTT G | 12 |
| TTG CCT CCG GTT CTG AAG GTG TTC TTG TAC | 13 |
| CTG TTG CCT CCG GTT CTG AAG GTG | 14 |
| CAT TCA ACT GTT GCC TCC GGT TCT GAA GGT G | 15 |
| CTG TTG CCT CCG GTT CTG | 16 |
| CAA TGC CAT CCT GGA GTT CCT G | 17 |
| GCT GCC CAA TGC CAT CCT GGA GTT CCT GTA AGA | 18 |
| T | |
| GCT GCC CAA TGC CAT CCT GGA GTT CCT G | 19 |
| GCT GCC CAA TGC CAT CCT GGA GTT CCT GTA A | 20 |
| CAA TGC CAT CCT GGA GTT CCT GTA AGA TAC C | 21 |
| CAA TGC CAT CCT GGA GTT CCT GTA AGA T | 22 |
| TGC CAT CCT GGA GTT CCT GTA AGA TAC C | 23 |
| GCT GCC CAA TGC CAT CCT GGA GTT CCT GTA AGA | 24 |
| TAC CAA | |
| CAA TGC CAT CCT GGA GTT CCT GTA AGA | 25 |
| GCT GCC CAA TGC CAT CCT GGA GTT CCT GTA AG | 26 |
| GCC CAA TGC CAT CCT GGA GTT CCT GTA AGA TAC | 27 |
| C | |
| GCC CAA TGC CAT CCT GGA GTT CCT GTA AGA T | 28 |
| TTG CCG CTG CCC AAT GCC ATC CTG GAG TTC CTG | 29 |
| TAA GAT | |
| GCC CAA TGC CAT CCT GGA GTT CCT GTA A | 30 |
| GCC GCT GCC CAA TGC CAT CCT GGA GTT CCT GTA A | 31 |
| GCC CAA TGC CAT CCT GGA GTT CCT G | 32 |
| GCC GCT GCC CAA TGC CAT CCT GGA GTT CCT G | 33 |
| m5C-G-m5C-T-G-C-m5C-m5C-A-A-T-G-m5C-m5C-A-U- | 34 |
| m5C-m5C | |
| GAT CTG TCA AAT CGC CTG CAG GTA A | 35 |
| GAT CTG TCA AAT CGC CTG CAG G | 36 |
| CAG ATC TGT CAA ATC GCC TGC AGG | 37 |
| GAT CTG TCA AAT CGC CTG CAG GT | 38 |
| GAT CTG TCA AAT CGC CTG CAG | 39 |
| CAG ATC TGT CAA ATC GCC TGC AGG T | 40 |
| CAG ATC TGT CAA ATC GCC TGC AG | 41 |
| GGG ATC CAG TAT ACT TAC AGG C | 42 |
| GGG ATC CAG TAT ACT TAC AGG CTC C | 43 |
| GAT CCA GTA TAC TTA CAG GCT CC | 44 |
| GGA TCC AGT ATA CTT ACA GGC TCC | 45 |
| ACT TCC TCT TTA ACA GAA AAG CAT AC | 46 |
| ATC CAG TAT ACT TAC AGG CTC C | 47 |
| GAG CTC AGA TCT TCT AAC TTC CTC T | 48 |
| GGG ATC CAG TAT ACT TAC AGG CTC | 49 |
| ATG GGA TCC AGT ATA CTT ACA GGC TCC | 50 |
| CTG TTC CAA ATC CTG CAT TGT TGC C | 51 |

wherein A is

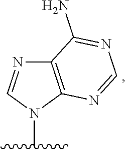 [SMILES: *n1cnc2c(N)ncnc21]

### C is

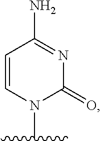 [SMILES: *n1ccc(N)nc1=O]

### G is

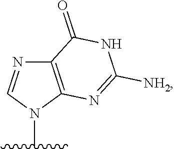 [SMILES: *n1cnc2c(=O)[nH]c(N)nc21]

### T is

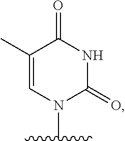 [SMILES: *n1cc(C)c(=O)[nH]c1=O]

### U is

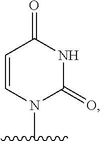 [SMILES: *n1ccc(=O)[nH]c1=O]

Gm is methylated guanine, Am is methylated adenine, and m5C is

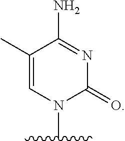 [SMILES: *n1cc(C)c(N)nc1=O]

In some aspects, each Nu from 1 to n and 5′ to 3′ of the antisense oligonucleotide conjugate of Formula (III), or pharmaceutically acceptable salt thereof, in the composition corresponds to SEQ ID NO: 1, SEQ ID NO: 7, SEQ ID NO: 17, or SEQ ID NO: 51. In some aspects, each Nu from 1 to n and 5′ to 3′ of the antisense oligonucleotide of Formula (III), or pharmaceutically acceptable salt thereof, in the composition corresponds to SEQ ID NO: 1. In some aspects, each Nu from 1 to n and 5′ to 3′ of the antisense oligonucleotide of Formula (III), or pharmaceutically acceptable salt thereof, in the composition corresponds to SEQ ID NO: 7. In some aspects, each Nu from 1 to n and 5′ to 3′ of the antisense oligonucleotide of Formula (III), or pharmaceutically acceptable salt thereof, in the composition corresponds to SEQ ID NO: 17. In some aspects, each Nu from 1 to n and 5′ to 3′ of the antisense oligonucleotide of Formula (III), or pharmaceutically acceptable salt thereof, in the composition corresponds to SEQ ID NO: 51.

In some aspects, the antisense oligonucleotide conjugate in the composition is PPMO #1, PPMO #2, PPMO #3, or PPMO #4, or a pharmaceutically acceptable salt thereof. In some aspects, the antisense oligonucleotide conjugate in the composition is PPMO #1, or a pharmaceutically acceptable salt thereof. In some aspects, the antisense oligonucleotide conjugate in the composition is PPMO #2, or a pharmaceutically acceptable salt thereof. In some aspects, the antisense oligonucleotide conjugate in the composition is PPMO #3, or a pharmaceutically acceptable salt thereof. In some aspects, the antisense oligonucleotide conjugate in the composition is PPMO #4, or a pharmaceutically acceptable salt thereof.

In some aspects, the antisense oligonucleotide conjugate in the composition is in free base form. In some aspects, the antisense oligonucleotide conjugate in the composition is a pharmaceutically acceptable salt. In some aspects, the antisense oligonucleotide conjugate in the composition is a halide salt. In some aspects, the antisense oligonucleotide conjugate is an HCl salt. In some aspects, the HCl salt of the antisense oligonucleotide conjugate is a 1HCl, 2HCl, 3HCl, 4HCl, 5HCl, or 6HCl salt.

In some aspects, the antisense oligonucleotide conjugate in the composition is the 6HCl salt form of PPMO #1, PPMO #2, PPMO #3, or PPMO #4. In some aspects, the antisense oligonucleotide conjugate in the composition is the 6HCl salt form of PPMO #1. In some aspects, the antisense oligonucleotide conjugate in the composition is the 6HCl salt form of PPMO #2. In some aspects, the antisense oligonucleotide conjugate in the composition is the 6HCl salt form of PPMO #3. In some aspects, the antisense oligonucleotide conjugate in the composition is the 6HCl salt form of PPMO #4.

In some aspects, the one or more surfactants are present in the composition in an amount between about 0.001% and about 2% by weight of the composition. In some aspects, the one or more surfactants are present in the composition in an amount between about 0.002% and about 1% by weight of the composition. In some aspects, the one or more surfactants in the composition are selected from the group consisting of polyoxyethylene sorbitan monooleate (polysorbate 80, polysorbate 20), polyvinylpyrrolidone (PVP, povidone, poloxamer, poloxamer F188), and mixtures thereof.

In some aspects, the one or more sugars are present in an amount between about 0.1% and about 11% by weight of the composition. In some aspects, the one or more sugars are present in an amount of about 6% by weight. In some aspects, the one or more sugars are selected from the group consisting of trehalose, mannitol, sucrose, and mixtures thereof. In some aspects, the sugar is a mixture of mannitol and sucrose. In some aspects, the sugar is mannitol. In some aspects, the sugar is sucrose.

In some aspects, the one or more buffering agents are present in an amount sufficient to provide a pH of about 6.0 to about 7.0 when the composition is dissolved or dispersed in an aqueous solvent. In some aspects, the one or more buffering agents is present in an amount to provide a pH of about 6.3 to about 6.5. In some aspects, the one or more buffering agents are present in a range of about 10 mM to about 50 mM when the composition is dissolved or dispersed in an aqueous solvent. In some aspects, the one or more buffering agents are selected from the group consisting of histidine, citrate, maleate, arginine and mixtures thereof. In some aspects, the buffering agent is citrate.

In some aspects, the composition has an osmolality between about 250 mOsm/kg and about 650 mOsm/kg. In some aspects, the composition has an osmolality between about 300 mOsm/kg and about 500 mOsm/kg. In some aspects, the composition has an osmolality between about 400 mOsm/kg and about 600 mOsm/kg.

In some aspects, the composition is in lyophilized form. In some aspects, the composition dissolves within 30 minutes after addition of an aqueous solvent. In some aspects, the composition dissolves within 15 minutes after addition of an aqueous solvent. In some aspects, the composition dissolves within 3 minutes after addition of an aqueous solvent.

In some aspects, the aqueous solvent used to dissolve the composition is sterile water.

A method of treating a human patient having Duchenne muscular dystrophy, comprising dissolving or dispersing a composition disclosed herein in an aqueous solvent is also disclosed herein.

In some aspects, the amount of aqueous solvent used to dissolve or disperse the composition is sufficient to provide a concentration of the antisense oligonucleotide conjugate of about 20 mg/mL to about 200 mg/mL. In some aspects, the amount of aqueous solvent used to dissolve or disperse the composition is sufficient to provide a concentration of the antisense oligonucleotide conjugate of about 20 mg/mL to about 100 mg/mL. In some aspects, the amount of aqueous solvent used to dissolve or disperse the composition is sufficient to provide a concentration of the antisense oligonucleotide conjugate of about 50 mg/mL. In some aspects, the amount of aqueous solvent used to dissolve or disperse the composition is sufficient to provide a concentration of the antisense oligonucleotide conjugate of about 100 mg/mL.

### DETAILED DESCRIPTION OF THE DISCLOSURE

Aspects of the present disclosure relate generally to pharmaceutical compositions comprising antisense oligonucleotide conjugates that are specifically designed to induce exon skipping in the human dystrophin gene, and methods of use thereof. Dystrophin plays a vital role in muscle function, and various muscle-related diseases are characterized by mutated forms of this gene, such as the mutated dystrophin genes found in Duchenne muscular dystrophy (DMD) and Becker muscular dystrophy (BMD).

Due to aberrant mRNA splicing events caused by mutations, these mutated human dystrophin genes either express defective dystrophin protein or express no measurable dystrophin at all, a condition that leads to various forms of muscular dystrophy. To remedy this condition, the antisense oligonucleotide conjugates in the pharmaceutical compositions of the present disclosure hybridize to selected regions of a pre-processed mRNA of a mutated human dystrophin gene, induce exon skipping and differential splicing in that otherwise aberrantly spliced dystrophin mRNA, and thereby allow muscle cells to produce an mRNA transcript that encodes a functional dystrophin protein. In certain aspects, the resulting dystrophin protein is not necessarily the “wild-type” form of dystrophin, but is rather a truncated, yet functional, form of dystrophin.

By increasing the levels of functional dystrophin protein in muscle cells, these and related aspects are useful in the prophylaxis and treatment of muscular dystrophy, especially those forms of muscular dystrophy, such as DMD and BMD, that are characterized by the expression of defective dystrophin proteins due to aberrant mRNA splicing. The specific antisense oligonucleotide conjugates included in the pharmaceutical composition described herein further provide dystrophin-exon-specific targeting over other oligonucleotides.

Thus, the disclosure relates to pharmaceutical compositions comprising: (a) an antisense oligonucleotide conjugate comprising a cell penetrating peptide covalently attached to a nucleic acid analog, or a pharmaceutically acceptable salt thereof, wherein the cell penetrating peptide (“CPP”) includes at least two positively charged amino acids; (b) one or more surfactants; (c) one or more sugars; and (d) one or more buffering agents.

In some aspects, the CPP is an arginine-rich peptide. The term “arginine-rich” refers to a CPP having at least 2, and preferably 2, 3, 4, 5, 6, 7, or 8 arginine residues, each optionally separated by one or more uncharged, hydrophobic residues, and optionally containing about 6-14 amino acid residues. As explained below, a CPP is linked at its carboxy terminus to the 3′ end of an antisense oligonucleotide through a linker, which can also be one or more amino acids, and is preferably also capped at its amino terminus by a substituent R^{a} with R^{a} selected from H, acyl, acetyl, benzoyl, or stearoyl. In some aspects, R^{a} is acetyl.

As seen in the table below, non-limiting examples of CPP's for use herein include —(RXR)₄—R^{a} (SEQ ID NO: 54), R-(FFR)₃-R^{a} (SEQ ID NO: 55), —B—X—(RXR)₄—R^{a} (SEQ ID NO: 56), —B—X—R-(FFR)₃-R^{a} (SEQ ID NO: 57), -GLY-R-(FFR)₃-R^{a} (SEQ ID NO: 58), -GLY-R₅—R^{a} (SEQ ID NO: 59), —R₅—R^{a} (SEQ ID NO: 60), -GLY-R₆—R^{a} (SEQ ID NO: 52) and —R₆—R^{a} (SEQ ID NO: 53), wherein R^{a} is selected from H, acyl, benzoyl, and stearoyl, and wherein R is arginine, X is 6-aminohexanoic acid, B is β-alanine, F is phenylalanine and GLY (or G) is glycine. The CPP “R₅” is meant to indicate a peptide of five (5) arginine residues linked together via amide bonds (and not a single substituent e.g., R⁵) (“R₅” disclosed as SEQ ID NO: 60). The CPP “R₆” is meant to indicate a peptide of six (6) arginine residues linked together via amide bonds (and not a single substituent e.g. R⁶) (“R₆” disclosed as SEQ ID NO: 53). In some aspects, R^{a} is acetyl.

Exemplary CPPs are provided in Table 1 (SEQ ID NOS: 52-60).

TABLE 1
Exemplary Cell penetrating Peptides

| Name | Sequence | SEQ ID NO. |
|---|---|---|
| R₆G | RRRRRRG | 52 |
| R₆ | RRRRRR | 53 |
| (RXR)₄ | RXRRXRRXRRXR | 54 |
| (RFF)₃R | RFFRFFRFFR | 55 |
| (RXR)₄XB | RXRRXRRXRRXRXB | 56 |
| (RFF)₃RXB | RFFRFFRFFRXB | 57 |
| (RFF)₃RG | RFFRFFRFFRG | 58 |
| R₅G | RRRRRG | 59 |
| R₅ | RRRRR | 60 |

R is arginine; X is 6-aminohexanoic acid; B is ß-alanine; F is phenylalanine; G is glycine

CPPs, their synthesis, and methods of conjugating to an oligonucleotide are further described in U.S. Application Publication No. US 2012/0289457 and International Patent Application Publication Nos. WO 2004/097017, WO 2009/005793, and WO 2012/150960, the disclosures of which are incorporated herein by reference in their entirety.

In some aspects, an antisense oligonucleotide comprises a substituent “Z,” defined as the combination of a CPP and a linker. The linker bridges the CPP at its carboxy terminus to the 3′-end of the oligonucleotide.

The linker within Z can comprise, for example, 1, 2, 3, 4, or 5 amino acids.

In particular aspects, Z is selected from:
—C(O)(CH₂)₅NH-CPP;
—C(O)(CH₂)₂NH-CPP;
—C(O)(CH₂)₂NHC(O)(CH₂)₅NH-CPP;
—C(O)CH₂NH-CPP, and the formula:

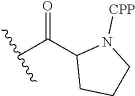

wherein the CPP is attached to the linker moiety by an amide bond at the CPP carboxy terminus.

In various aspects, the CPP is an arginine-rich peptide as described herein and seen in Table 1. In certain aspects, the arginine-rich CPP is —R₆—R^{a} (SEQ ID NO: 53), (i.e., six arginine residues; SEQ ID NO. 53), wherein Ra is selected from H, acyl, acetyl, benzoyl, and stearoyl. In certain aspects, R^{a} is acetyl. In various aspects, the CPP is selected from SEQ ID NOS: 53, 54, or 55, and the linker is selected from the group consisting of —C(O)(CH₂)₅NH—, —C(O)(CH₂)₂NH—, —C(O)(CH₂)₂NHC(O)(CH₂)₅NH—, —C(O)CH₂NH—, and

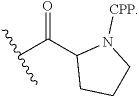

In some aspects, the linker comprises 1, 2, 3, 4, or 5 amino acids.

In some aspects, the CPP is SEQ ID NO. 53 and the linker is Gly. In some aspects, the CPP is SEQ ID NO. 52.

In certain aspects, Z is —C(O)CH₂NH—R₆—R^{a} (SEQ ID NO: 52) covalently bonded to an antisense oligonucleotide at the 3′ end of the oligonucleotide, wherein R^{a} is H, acyl, acetyl, benzoyl, or stearoyl to cap the amino terminus of the R₆ (SEQ ID NO: 53). In certain aspects, R^{a} is acetyl. In these non-limiting examples, the CPP is —R₆—R^{a} (SEQ ID NO: 53) and the linker is —C(O)CH₂NH—, (i.e. GLY). This particular example of Z=—C(O)CH₂NH—R₆—R^{a} (SEQ ID NO: 52) is also exemplified by the following structure:

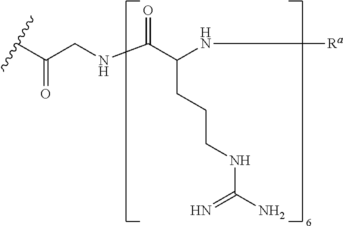

wherein R^{a} is selected from H, acyl, acetyl, benzoyl, and stearoyl.

In various aspects, the CPP is —R₆—R^{a} (SEQ ID NO: 53), also exemplified as the following formula:

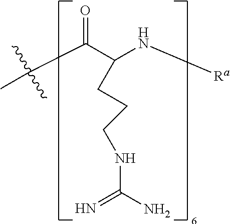

wherein R^{a} is selected from H, acyl, acetyl, benzoyl, and stearoyl. In certain aspects, the CPP is SEQ ID NO. 52. In some aspects, R^{a} is acetyl.

In some aspects, the CPP is —(RXR)₄—R^{a} (SEQ ID NO. 54), also exemplified as the following formula:

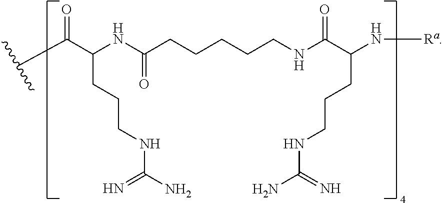

In various aspects, the CPP is —R-(FFR)₃-R^{a} (SEQ ID NO. 55), also exemplified as the following formula:

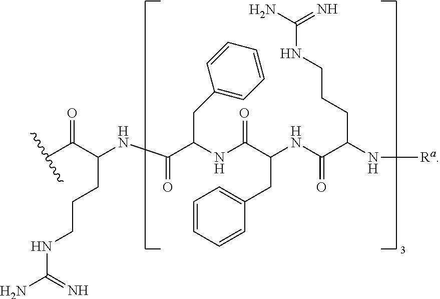

In various aspects, Z is selected from:
—C(O)(CH₂)₅NH-CPP;
—C(O)(CH₂)₂NH-CPP;
—C(O)(CH₂)₂NHC(O)(CH₂)₅NH-CPP;
—C(O)CH₂NH-CPP; and the formula:

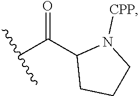

wherein the CPP is attached to the linker moiety by an amide bond at the CPP carboxy terminus, and wherein the CPP is selected from:

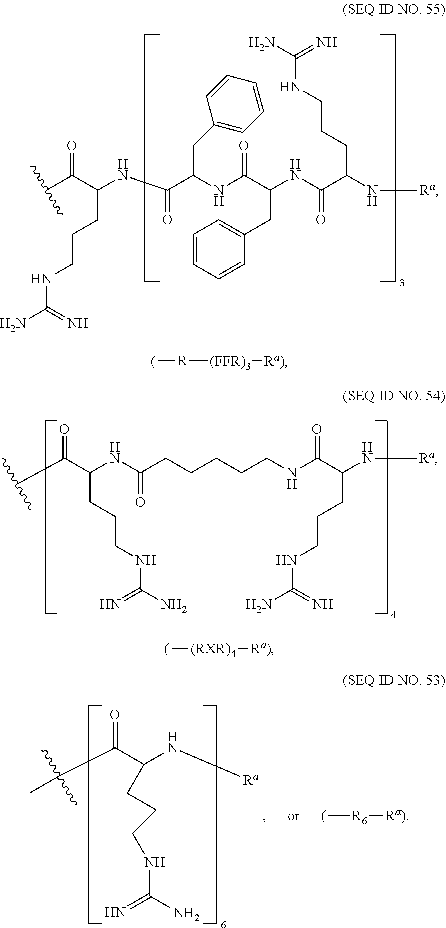

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described. For the purposes of the present disclosure, the following terms are defined below.

### I. Definitions

The term “alkyl,” as used herein, unless otherwise specified, refers to a saturated straight or branched hydrocarbon. In certain aspects, the alkyl group is a primary, secondary, or tertiary hydrocarbon. In certain aspects, the alkyl group includes one to ten carbon atoms, i.e., C₁ to C₁₀ alkyl. In certain aspects, the alkyl group includes one to six carbon atoms, i.e., C₁ to C₆ alkyl. The term includes both substituted and unsubstituted alkyl groups, including halogenated alkyl groups. In certain aspects, the alkyl group is a fluorinated alkyl group. Non-limiting examples of moieties with which the alkyl group can be substituted are selected from the group consisting of halogen (fluoro, chloro, bromo, or iodo), hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference. In certain aspects, the alkyl group is selected from the group consisting of methyl, CF₃, CCl₃, CFCl₂, CF₂Cl, ethyl, CH₂CF₃, CF₂CF₃, propyl, isopropyl, butyl, isobutyl, sec-butyl, t-butyl, pentyl, isopentyl, neopentyl, hexyl, isohexyl, 3-methylpentyl, 2,2-dimethylbutyl, and 2,3-dimethylbutyl.

“Amenable to exon skipping” as used herein with regard to a subject or patient is intended to include subjects and patients having one or more mutations in the dystrophin gene which, absent the skipping of the specified exon or intron of the dystrophin pre-mRNA, causes the reading frame to be out-of-frame thereby disrupting translation of the pre-mRNA leading to an inability of the subject or patient to produce functional or semi-functional dystrophin. Determining whether a patient has a mutation in the dystrophin gene that is amenable to exon skipping is well within the purview of one of skill in the art (see, e.g., Aartsma-Rus et al. (2009) Hum Mutat. 30:293-299; Gurvich et al., Hum Mutat. 2009; 30(4) 633-640; and Fletcher et al. (2010) Molecular Therapy 18(6) 1218-1223.).

The term “nucleic acid analog” or “polynucleic acid analog” as used herein is a compound that is structurally similar to naturally occurring RNA. A nucleic acid analog or polynucleic acid analog is a chain of nucleotides, in which the phosphate backbone, pentose sugar, and/or nucleobase is modified as compared with naturally occurring RNA. An exemplary nucleic acid analog includes a peptide nucleic acid (PNA), morpholino, and locked nucleic acid (LNA).

The term “oligonucleotide” as used herein refers to a sequence of subunits connected by intersubunit linkages. In certain instances, the term “oligonucleotide” is used in reference to an “antisense oligonucleotide.” For “antisense oligonucleotides,” each subunit consists of: (i) a ribose sugar or a derivative thereof; and (ii) a nucleobase bound thereto, such that the order of the base-pairing moieties forms a base sequence that is complementary to a target sequence in a nucleic acid (typically an RNA) by Watson-Crick base pairing, to form a nucleic acid: oligonucleotide heteroduplex within the target sequence with the proviso that either the subunit, the intersubunit linkage, or both are not naturally occurring. In certain aspects, the “antisense oligonucleotide conjugate” is a phosphorodiamidate morpholino antisense oligonucleotide in which a CPP is attached to the 3′ terminus of the antisense oligonucleotide (“PPMO”).

The terms “complementary” and “complementarity” refer to two or more oligonucleotides (i.e., each comprising a nucleobase sequence) that are related with one another by Watson-Crick base-pairing rules. For example, the nucleobase sequence “T-G-A (5′→3′),” is complementary to the nucleobase sequence “A-C-T (3′→5′).” Complementarity can be “partial,” in which less than all of the nucleobases of a given nucleobase sequence are matched to the other nucleobase sequence according to base pairing rules. For example, in some aspects, complementarity between a given nucleobase sequence and the other nucleobase sequence can be about 70%, about 75%, about 80%, about 85%, about 90% or about 95%. Or, there can be “complete” or “perfect” (100%) complementarity between a given nucleobase sequence and the other nucleobase sequence to continue the example. The degree of complementarity between nucleobase sequences has significant effects on the efficiency and strength of hybridization between the sequences.

“Enhance” or “enhancing,” or “increase” or “increasing,” or “stimulate” or “stimulating” refers generally to the ability of one or more antisense oligonucleotide conjugates or pharmaceutical compositions of any of the foregoing to produce or cause a greater physiological response (i.e., downstream effects) in a cell or a subject, as compared to the response caused by either no antisense oligonucleotide conjugate or a control compound. A greater physiological response can include increased expression of a functional form of a dystrophin protein, or increased dystrophin-related biological activity in muscle tissue, among other responses apparent from the understanding in the art and the description herein.

As used herein, the terms “function” and “functional” and the like refer to a biological, enzymatic, or therapeutic function.

A “functional” dystrophin protein refers generally to a dystrophin protein having sufficient biological activity to reduce the progressive degradation of muscle tissue that is otherwise characteristic of muscular dystrophy, typically as compared to the altered or “defective” form of dystrophin protein that is present in certain subjects with DMD or BMD. As one example, dystrophin-related activity in muscle cultures in vitro can be measured according to myotube size, myofibril organization (or disorganization), contractile activity, and spontaneous clustering of acetylcholine receptors (see, e.g., Brown et al., Journal of Cell Science. 112:209-216, 1999). Animal models are also valuable resources for studying the pathogenesis of disease, and provide a means to test dystrophin-related activity. Two of the most widely used animal models for DMD research are the mdx mouse and the golden retriever muscular dystrophy (GRMD) dog, both of which are dystrophin negative (see, e.g., Collins & Morgan, Int J Exp Pathol 84: 165-172, 2003). These and other animal models can be used to measure the functional activity of various dystrophin proteins. Included are truncated forms of dystrophin, such as those forms that are produced following the administration of certain of the exon-skipping antisense oligonucleotide conjugates of the present disclosure.

The terms “mismatch” or “mismatches” refer to one or more nucleobases (whether contiguous or separate) in an oligonucleotide nucleobase sequence that are not matched to a target pre-mRNA according to base pairing rules. While perfect complementarity is often desired, some aspects can include one or more but preferably 6, 5, 4, 3, 2, or 1 mismatches with respect to the target pre-mRNA. Variations at any location within the oligonucleotide are included. In certain aspects, antisense oligonucleotide conjugates of the disclosure include variations in nucleobase sequence near the termini, variations in the interior, and if present are typically within about 6, 5, 4, 3, 2, or 1 subunits of the 5′ and/or 3′ terminus. In certain aspects, one, two, or three nucleobases can be removed and still provide on-target binding.

In certain aspects, a morpholino is conjugated at the 5′ end of the oligonucleotide with a “tail” moiety to increase its stability and/or solubility. Exemplary tails include:

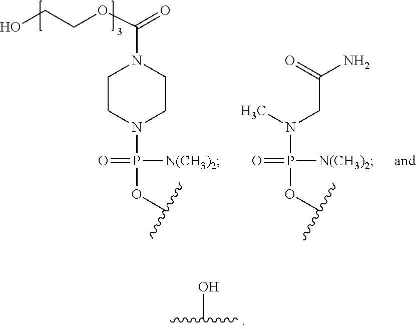

Of the above exemplary tail moieties, “TEG” or “EG3” refers to the following tail moiety:

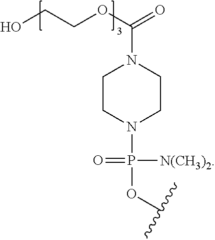

Of the above exemplary tail moieties, “GT” refers to the following tail moiety:

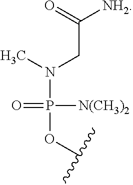 [SMILES: *OP(=O)(N(C)C)N(C)CC(N)=O]

As used herein, the terms “-G-R₅” (SEQ ID NO: 59) and “-G-R₅—Ac” (SEQ ID NO: 59) are used interchangeably and refer to a cell penetrating peptide moiety conjugated to an antisense oligonucleotide. In various aspects, “G” represents a glycine residue conjugated to “R₅” by an amide bond, and each “R” represents an arginine residue conjugated together by amide bonds such that “R₅” means five (5) arginine residues conjugated together by amide bonds (“R₅” disclosed as SEQ ID NO: 60). The arginine residues can have any stereo configuration, for example, the arginine residues can be L-arginine residues, D-arginine residues, or a mixture of D- and L-arginine residues. In some aspects, “-G-R₅” (SEQ ID NO: 59) or “-G-R₅—Ac” (SEQ ID NO: 59) is conjugated to the 3′ end of an antisense oligonucleotide and is of the following formula:

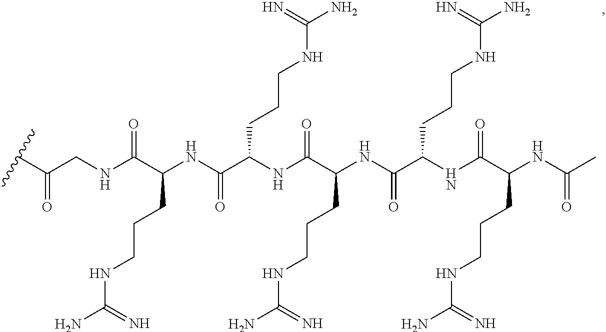 [SMILES: *C(=O)CNC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(C)=O]

or a pharmaceutically acceptable salt thereof, or

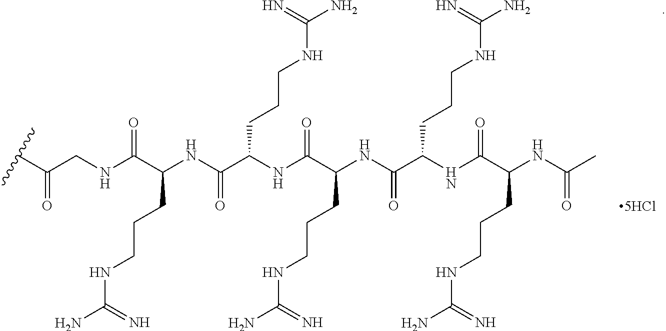 [SMILES: *C(=O)CNC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(C)=O ; "·5HCl"]

As used herein, the terms “-G-R₆” (SEQ ID NO: 52) and “-G-R₆—Ac” (SEQ ID NO: 52) and “R₆G” (SEQ ID NO: 52) are used interchangeably and refer to a cell penetrating peptide moiety conjugated to an antisense oligonucleotide. In various aspects, “G” represents a glycine residue conjugated to “R₆” by an amide bond, and each “R” represents an arginine residue conjugated together by amide bonds such that “R₆” means six (6) arginine residues conjugated together by amide bonds (“R₆” disclosed as SEQ ID NO: 53). The arginine residues can have any stereo configuration, for example, the arginine residues can be L-arginine residues, D-arginine residues, or a mixture of D- and L-arginine residues. In some aspects, “-G-R₆” (SEQ ID NO: 52) or “-G-R₆—Ac” (SEQ ID NO: 52) is conjugated to the 3′ end of an antisense oligonucleotide and is of the following formula:

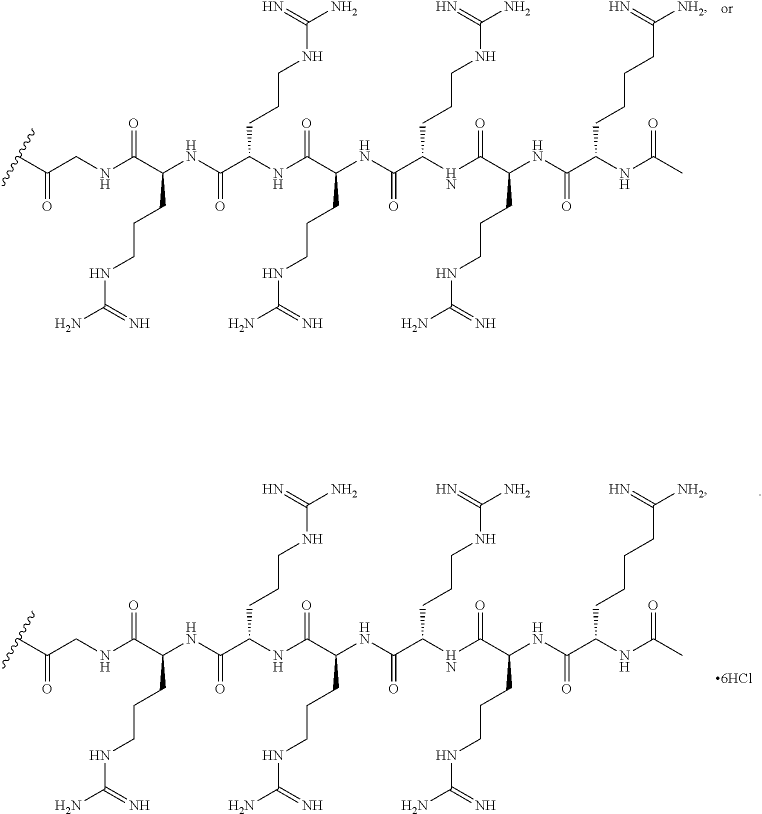 [SMILES: *C(=O)CNC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCCC(=N)N)NC(C)=O ; *C(=O)CNC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCCC(=N)N)NC(C)=O]

The terms “nucleobase” (Nu), “base pairing moiety” or “base” are used interchangeably to refer to a purine or pyrimidine base found in naturally occurring, or “native” DNA or RNA (e.g., uracil, thymine, adenine, cytosine, and guanine), as well as analogs of these naturally occurring purines and pyrimidines. These analogs can confer improved properties, such as binding affinity, to the oligonucleotide. Exemplary analogs include hypoxanthine (the base component of inosine); 2,6-diaminopurine; 5-methyl cytosine; C5-propynyl-modified pyrimidines; 10-(9-(aminoethoxy) phenoxazinyl) (G-clamp) and the like.

Further examples of base pairing moieties include, but are not limited to, uracil, thymine, adenine, cytosine, guanine and hypoxanthine (inosine) having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). The modified nucleobases disclosed in: Chiu and Rana, RNA, 2003, 9, 1034-1048; Limbach et al. Nucleic Acids Research, 1994, 22, 2183-2196; and Revankar and Rao, Comprehensive Natural Products Chemistry, vol. 7, 313; are also contemplated, the contents of which are incorporated herein by reference.

Further examples of base pairing moieties include, but are not limited to, expanded-size nucleobases in which one or more benzene rings has been added. Nucleic acid base replacements described in: the Glen Research catalog (www.glenresearch.com); Krueger A T et al., Acc. Chem. Res., 2007, 40, 141-150; Kool, E T, Acc. Chem. Res., 2002, 35, 936-943; Benner S. A., et al., Nat. Rev. Genet., 2005, 6, 553-543; Romesberg, F. E., et al., Curr. Opin. Chem. Biol., 2003, 7, 723-733; and Hirao, I., Curr. Opin. Chem. Biol., 2006, 10, 622-627; the contents of which are incorporated herein by reference, are contemplated as useful in the antisense oligonucleotide conjugates described herein. Examples of expanded-size nucleobases include those shown below, as well as tautomeric forms thereof.

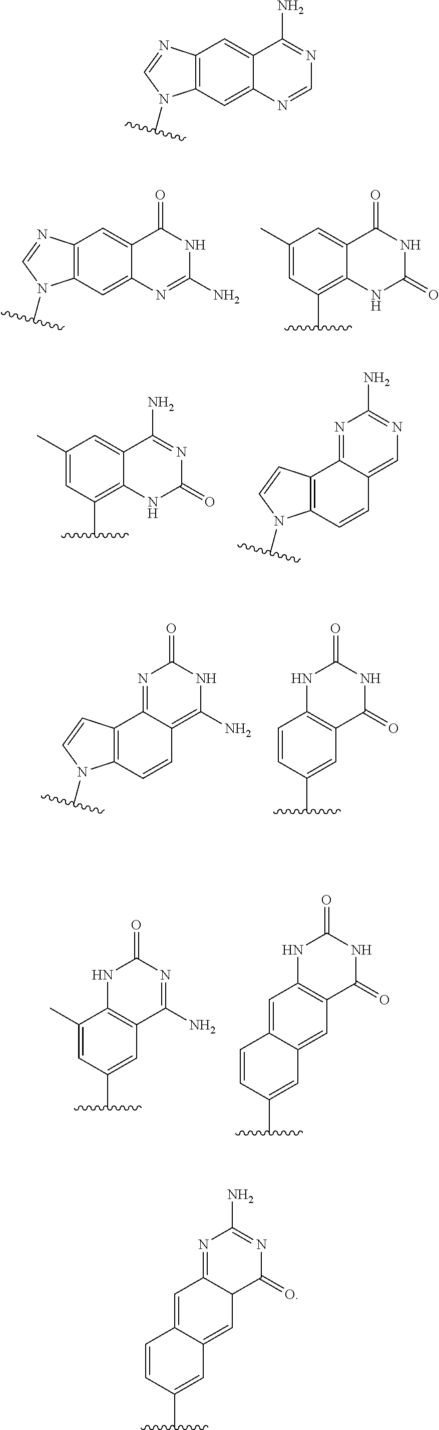

As used herein, a set of brackets used within a structural formula indicate that the structural feature between the brackets is repeated. In some aspects, the brackets used can be “[” and “],” and in certain aspects, brackets used to indicate repeating structural features can be “(” and “).” In some aspects, the number of repeat iterations of the structural feature between the brackets is the number indicated outside the brackets such as 2, 3, 4, 5, 6, 7, and so forth. In various aspects, the number of repeat iterations of the structural feature between the brackets is indicated by a variable indicated outside the brackets such as “Z”.

As used herein, a straight bond or a squiggly bond drawn to a chiral carbon or phosphorous atom within a structural formula indicates that the stereochemistry of the chiral carbon or phosphorous is undefined and is intended to include all forms of the chiral center and/or mixtures thereof. Examples of such illustrations are depicted below.

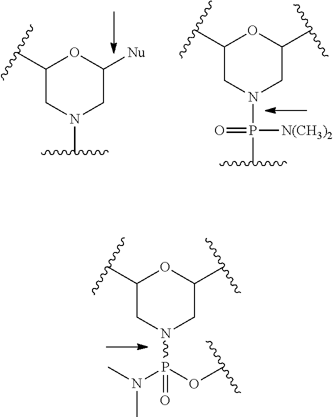

The phrase “pharmaceutically acceptable” means the substance or composition must be compatible, chemically and/or toxicologically, with the other ingredients comprising a formulation, and/or the subject being treated therewith.

The term “restoration” with respect to dystrophin synthesis or production refers generally to the production of a dystrophin protein including truncated forms of dystrophin in a patient with muscular dystrophy following treatment with an antisense oligonucleotide described herein. The percent of dystrophin-positive fibers in a patient following treatment can be determined by a muscle biopsy using known techniques. For example, a muscle biopsy can be taken from a suitable muscle, such as the biceps brachii muscle in a patient.

Analysis of the percentage of positive dystrophin fibers can be performed pre-treatment and/or post-treatment or at time points throughout the course of treatment. In some aspects, a post-treatment biopsy is taken from the contralateral muscle from the pre-treatment biopsy. Pre- and post-treatment dystrophin expression analysis can be performed using any suitable assay for dystrophin. In some aspects, immunohistochemical detection is performed on tissue sections from the muscle biopsy using an antibody that is a marker for dystrophin, such as a monoclonal or a polyclonal antibody. For example, the MANDYS106 antibody can be used which is a highly sensitive marker for dystrophin. Any suitable secondary antibody can be used.

In some aspects, the percent dystrophin-positive fibers are calculated by dividing the number of positive fibers by the total fibers counted. Normal muscle samples have 100% dystrophin-positive fibers. Therefore, the percent dystrophin-positive fibers can be expressed as a percentage of normal. To control for the presence of trace levels of dystrophin in the pretreatment muscle, as well as revertant fibers, a baseline can be set using sections of pre-treatment muscles from a patient when counting dystrophin-positive fibers in post-treatment muscles. This can be used as a threshold for counting dystrophin-positive fibers in sections of post-treatment muscle in that patient. In other aspects, antibody-stained tissue sections can also be used for dystrophin quantification using Bioquant image analysis software (Bioquant Image Analysis Corporation, Nashville, TN). The total dystrophin fluorescence signal intensity can be reported as a percentage of normal. In addition, Western blot analysis with monoclonal or polyclonal anti-dystrophin antibodies can be used to determine the percentage of dystrophin positive fibers. For example, the anti-dystrophin antibody NCL-Dys1 from Leica Biosystems can be used. The percentage of dystrophin-positive fibers can also be analyzed by determining the expression of the components of the sarcoglycan complex (β,γ) and/or neuronal NOS.

In some aspects, treatment with a pharmaceutical composition comprising an antisense oligonucleotide conjugate slows or reduces the progressive respiratory muscle dysfunction and/or failure in patients with DMD that would be expected without treatment. In some aspects, treatment with pharmaceutical composition comprising an antisense oligonucleotide conjugate can reduce or eliminate the need for ventilation assistance that would be expected without treatment. In some aspects, measurements of respiratory function for tracking the course of the disease, as well as the evaluation of potential therapeutic interventions include maximum inspiratory pressure (MIP), maximum expiratory pressure (MEP), and forced vital capacity (FVC). MIP and MEP measure the level of pressure a person can generate during inhalation and exhalation, respectively, and are sensitive measures of respiratory muscle strength. MIP is a measure of diaphragm muscle weakness.

In some aspects, MEP can decline before changes in other pulmonary function tests, including MIP and FVC. In certain aspects, MEP can be an early indicator of respiratory dysfunction. In certain aspects, FVC can be used to measure the total volume of air expelled during forced exhalation after maximum inspiration. In patients with DMD, FVC increases concomitantly with physical growth until the early teens. However, as growth slows or is stunted by disease progression, and muscle weakness progresses, the vital capacity enters a descending phase and declines at an average rate of about 8 to 8.5 percent per year after 10 to 12 years of age. In certain aspects, MIP percent predicted (MIP adjusted for weight), MEP percent predicted (MEP adjusted for age), and FVC percent predicted (FVC adjusted for age and height) are supportive analyses.

The terms “subject” and “patient” as used herein include any animal that exhibits a symptom, or is at risk for exhibiting a symptom, which can be treated with a pharmaceutical composition comprising an antisense oligonucleotide conjugate, such as a subject (or patient) that has or is at risk for having DMD or BMD, or any of the symptoms associated with these conditions (e.g., muscle fiber loss). Suitable subjects (or patients) include laboratory animals (such as mouse, rat, rabbit, or guinea pig), farm animals, and domestic animals or pets (such as a cat or dog). Non-human primates and, preferably, human patients (or subjects), are included. Also included are methods of producing dystrophin in a subject (or patient) having a mutation of the dystrophin gene that is amenable to exon 44, exon 45, exon 50, exon 51, exon 52, or exon 53 skipping.

The phase “targeting sequence” or “base sequence” refers to a sequence of nucleobases of an oligonucleotide that is complementary to a sequence of nucleotides in a target pre-mRNA. In some aspects of the disclosure, the sequence of nucleotides in the target pre-mRNA is an exon 44, exon 45, exon 50, exon 51, exon 52, or exon 53 target region of the dystrophin pre-mRNA.

“PPMO #1” has the following structure:

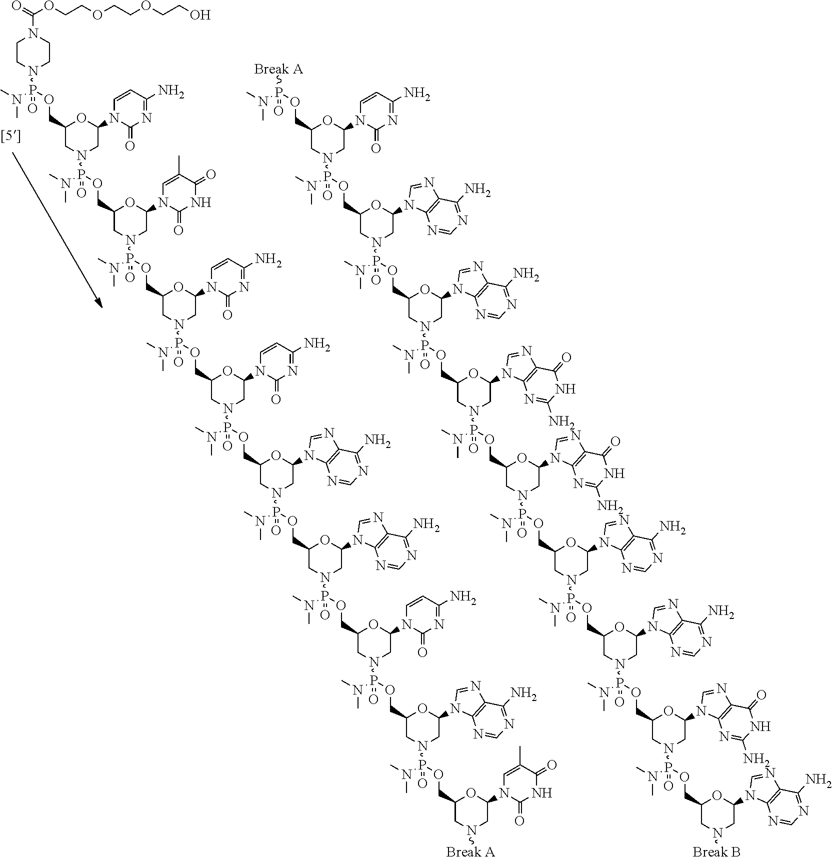

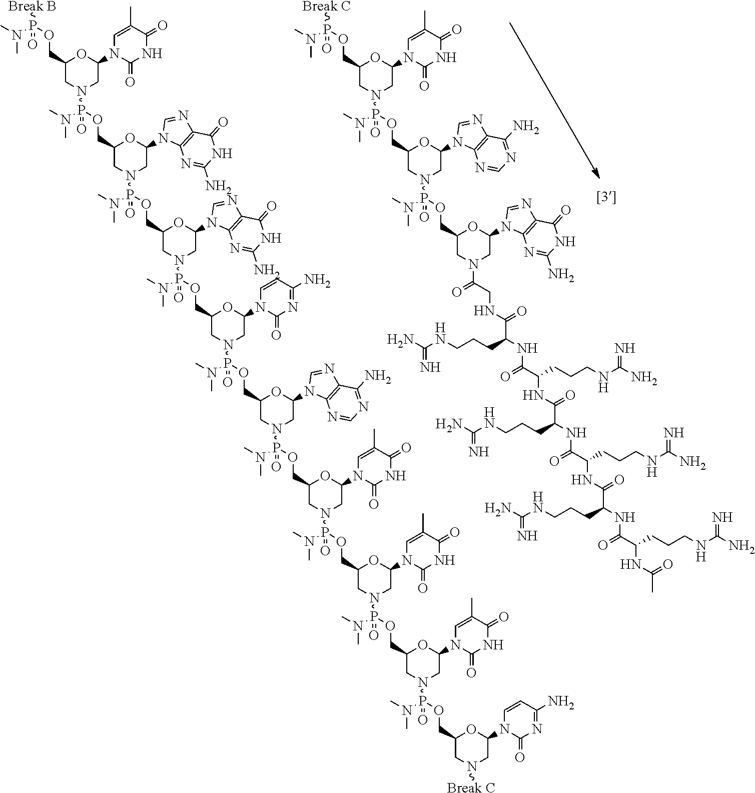

or a pharmaceutically acceptable salt thereof. In some aspects, PPMO #1 is in the form of a halide salt. In some aspects, PPMO #1 is in the form of a hexahalide salt form. In some aspects, PPMO #1 is in the form of an HCl (hydrochloric acid) salt. In certain aspects, the HCl salt is a ·6HCl salt.

“PPMO #2” is has the following structure:

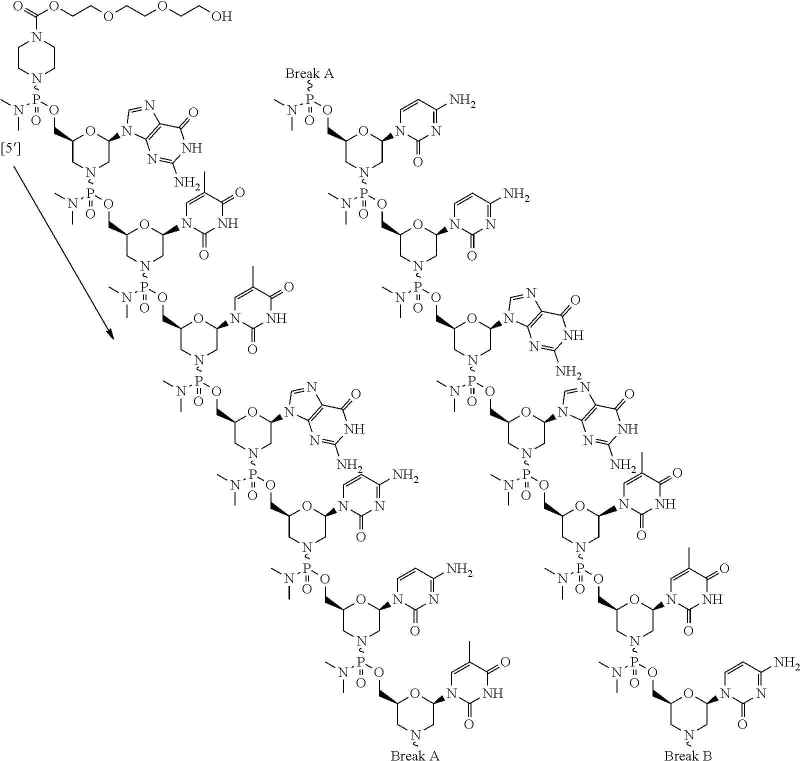

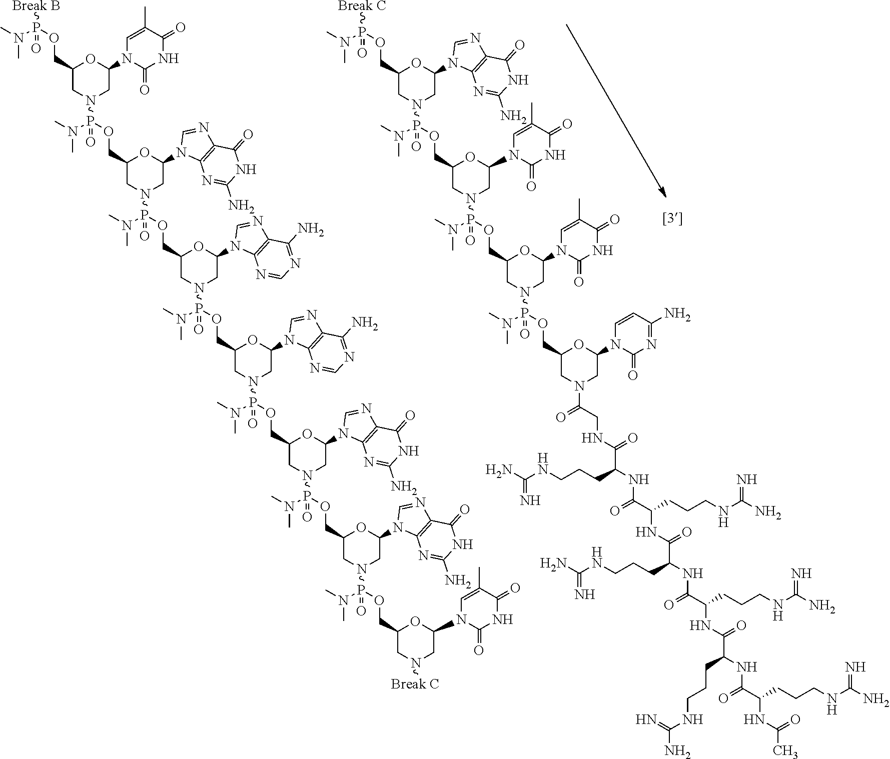

or a pharmaceutically acceptable salt thereof. In some aspects, PPMO #2 is in the form of a halide salt. In some aspects, PPMO #2 is in the form of a hexahalide salt form. In some aspects, PPMO #2 is in the form of an HCl (hydrochloric acid) salt. In certain aspects, the HCl salt is a ·6HCl salt.

“PPMO #3” is has the following structure:

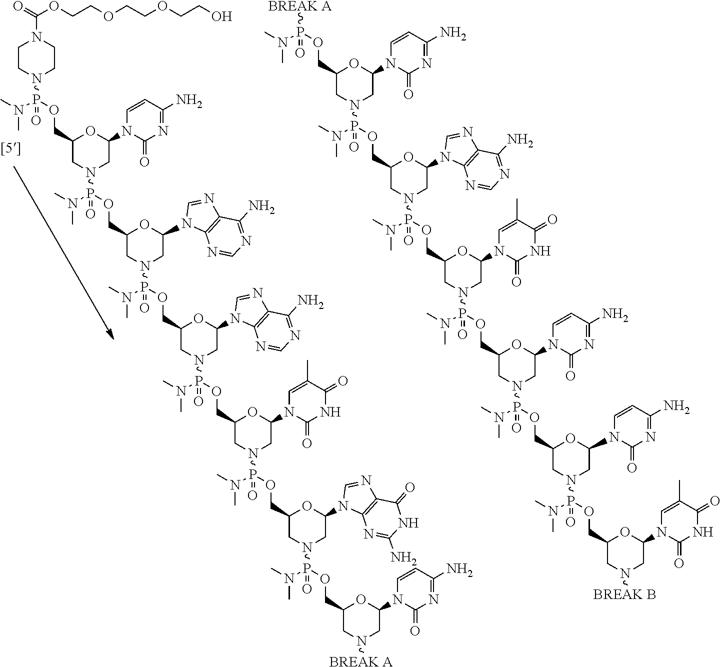

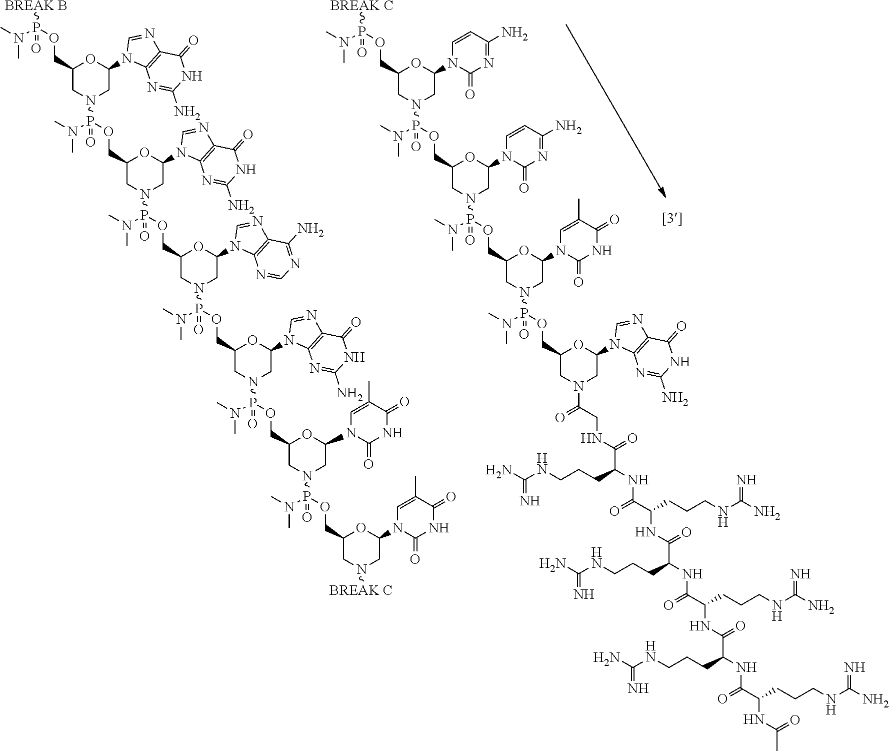

or a pharmaceutically acceptable salt thereof. In some aspects, PPMO #3 is in the form of a halide salt. In some aspects, PPMO #3 is in the form of a hexahalide salt form. In some aspects, PPMO #3 is in the form of an HCl (hydrochloric acid) salt. In certain aspects, the HCl salt is a ·6HCl salt.

“PPMO #4” is has the following structure:

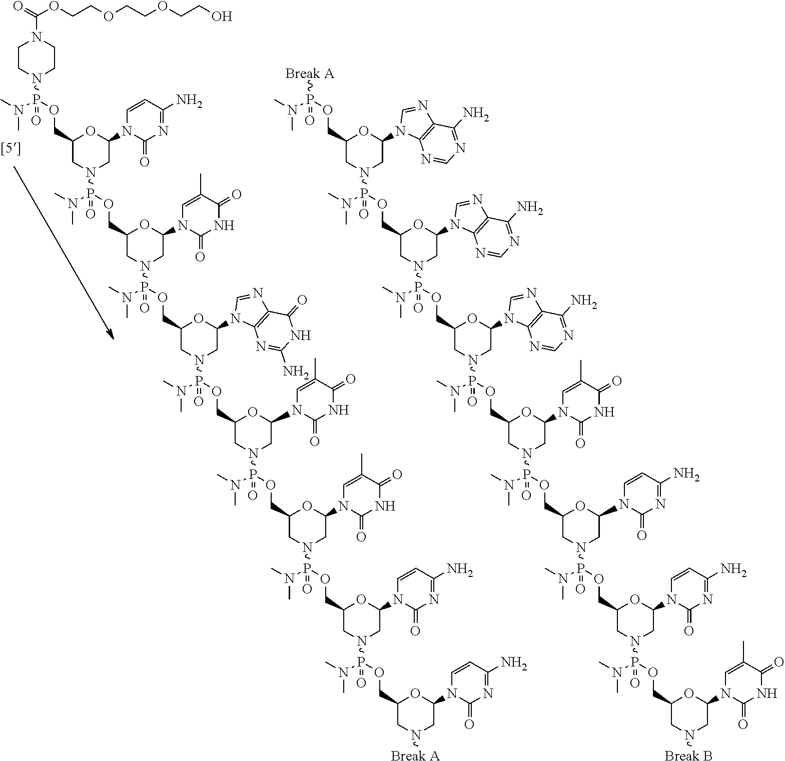

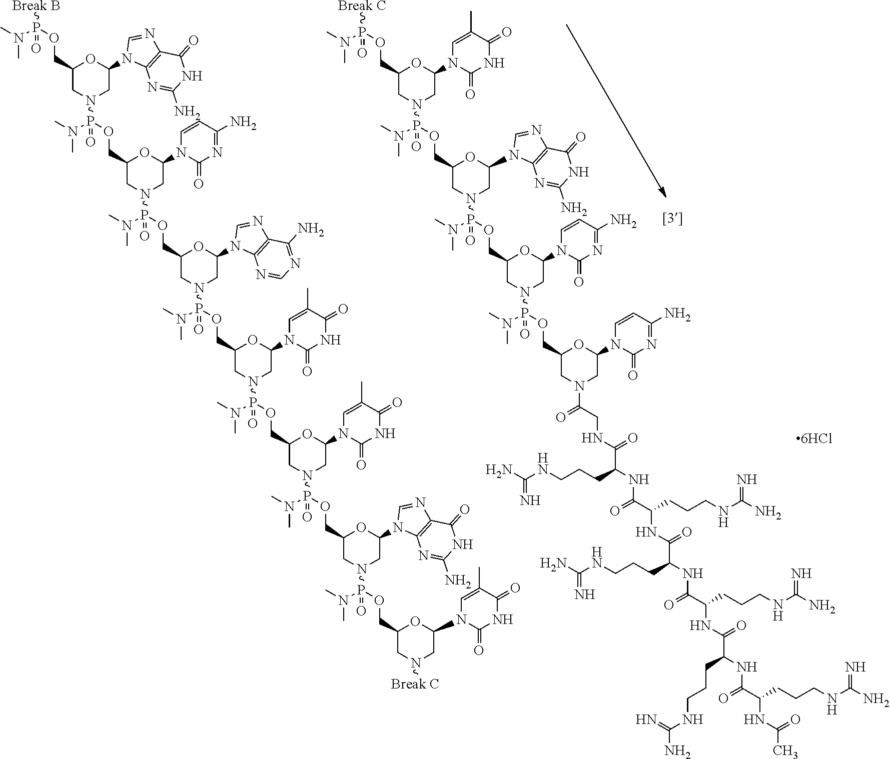

or a pharmaceutically acceptable salt thereof. In some aspects, PPMO #4 is in the form of a halide salt. In some aspects, PPMO #4 is in the form of a hexahalide salt form. In some aspects, PPMO #4 is in the form of an HCl (hydrochloric acid) salt. In certain aspects, the HCl salt is a ·6HCl salt.

“Treatment” of a subject (e.g. a mammal, such as a human) or a cell is any type of intervention used in an attempt to alter the natural course of the subject or cell. Treatment includes, but is not limited to, administration of an oligonucleotide or a pharmaceutical composition thereof, and can be performed either prophylactically or subsequent to the initiation of a pathologic event or contact with an etiologic agent. Treatment includes any desirable effect on the symptoms or pathology of a disease or condition associated with the dystrophin protein, as in certain forms of muscular dystrophy, and can include, for example, minimal changes or improvements in one or more measurable markers of the disease or condition being treated. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

In some aspects, treatment with a pharmaceutical composition comprising an antisense oligonucleotide conjugate increases novel dystrophin production, delays disease progression, slows or reduces the loss of ambulation, reduces muscle inflammation, reduces muscle damage, improves muscle function, reduces loss of pulmonary function, and/or enhances muscle regeneration that would be expected without treatment. In some aspects, treatment maintains, delays, or slows disease progression. In some aspects, treatment maintains ambulation or reduces the loss of ambulation. In some aspects, treatment maintains pulmonary function or reduces loss of pulmonary function. In some aspects, treatment maintains or increases a stable walking distance in a patient, as measured by, for example, the 6 Minute Walk Test (6MWT). In some aspects, treatment maintains or reduces the time to walk/run 10 meters (i.e., the 10 meter walk/run test). In some aspects, treatment maintains or reduces the time to stand from supine (i.e., time to stand test). In some aspects, treatment maintains or reduces the time to climb four standard stairs (i.e., the four-stair climb test). In some aspects, treatment maintains or reduces muscle inflammation in the patient, as measured by, for example, MRI (e.g., MRI of the leg muscles). In some aspects, MRI measures T2 and/or fat fraction to identify muscle degeneration. MRI can identify changes in muscle structure and composition caused by inflammation, edema, muscle damage, and fat infiltration.

In some aspects, treatment with a pharmaceutical composition comprising an antisense oligonucleotide conjugate increases novel dystrophin production and slows or reduces the loss of ambulation that would be expected without treatment. For example, treatment can stabilize, maintain, improve or increase walking ability (e.g., stabilization of ambulation) in the subject. In some aspects, treatment maintains or increases a stable walking distance in a patient, as measured by, for example, the 6 Minute Walk Test (6MWT), described by McDonald, et al. (Muscle Nerve, 2010; 42:966-74, herein incorporated by reference). A change in the 6 Minute Walk Distance (6MWD) can be expressed as an absolute value, a percentage change or a change in the %-predicted value. The performance of a DMD patient in the 6MWT relative to the typical performance of a healthy peer can be determined by calculating a %-predicted value. For example, the %-predicted 6MWD can be calculated using the following equation for males: 196.72+(39.81×age)−(1.36×age²)+(132.28×height in meters). For females, the %-predicted 6MWD can be calculated using the following equation: 188.61+(51.50×age)−(1.86×age²)+(86.10×height in meters) (Henricson et al. PLOS Curr., 2012, version 2, herein incorporated by reference).

Loss of muscle function in patients with DMD can occur against the background of normal childhood growth and development. Indeed, younger children with DMD can show an increase in distance walked during 6MWT over the course of about 1 year despite progressive muscular impairment. In some aspects, the 6MWD from patients with DMD is compared to typically developing control subjects and to existing normative data from age and sex matched subjects. In some aspects, normal growth and development can be accounted for using an age and height based equation fitted to normative data. Such an equation can be used to convert 6MWD to a percent-predicted (%-predicted) value in subjects with DMD. In certain aspects, analysis of %-predicted 6MWD data represents a method to account for normal growth and development, and can show that gains in function at early ages (e.g., less than or equal to age 7) represent stable rather than improving abilities in patients with DMD (Henricson et al. PLoS Curr., 2012, version 2, herein incorporated by reference).

An antisense molecule nomenclature system was proposed and published to distinguish between the different antisense molecules (see Mann et al., (2002) J Gen Med 4, 644-654). This nomenclature became especially relevant when testing several slightly different antisense molecules, all directed at the same target region, as shown below:
H#A/D(x:y).

The first letter designates the species (e.g. H: human, M: murine, C: canine). “#” designates target dystrophin exon number. “A/D” indicates acceptor or donor splice site at the beginning and end of the exon, respectively. (x y) represents the annealing coordinates where “−” or “+” indicate intronic or exonic sequences respectively. For example, A(−6+18) would indicate the last 6 bases of the intron preceding the target exon and the first 18 bases of the target exon. The closest splice site would be the acceptor so these coordinates would be preceded with an “A”. Describing annealing coordinates at the donor splice site could be D(+2−18) where the last 2 exonic bases and the first 18 intronic bases correspond to the annealing site of the antisense molecule. Entirely exonic annealing coordinates that would be represented by A(+65+85), that is the site between the 65th and 85th nucleotide from the start of that exon.

### II. Compositions

The disclosure relates to compositions comprising (a) an antisense oligonucleotide conjugate comprising a cell penetrating peptide covalently attached to a nucleic acid analog, or a pharmaceutically acceptable salt thereof, wherein the cell penetrating peptide includes at least two positively charged amino acids; (b) one or more surfactants; (c) one or more sugars; and (d) one or more buffering agents.

### A. Antisense Oligonucleotide Conjugates

### 1. Antisense Oligonucleotide Conjugates Designed to Induce Exon Skipping

In certain aspects, the antisense oligonucleotide conjugates, or pharmaceutically acceptable salts thereof, of the compositions of the disclosure are complementary to an exon 44, exon 45, exon 50, exon 51, exon 52, or exon 53 target region of the dystrophin pre-mRNA. In some aspects, the antisense oligonucleotide conjugates, or pharmaceutically acceptable salts thereof, of the composition of the disclosure are complementary to 15 to 35 nucleobases of an exon 44, exon 45, exon 50, exon 51, exon 52, or exon 53 target region of the dystrophin pre-mRNA. In some aspects, the antisense oligonucleotide conjugates, or pharmaceutically acceptable salts thereof, of the composition of the disclosure are complementary to 15, 16, 17, 18, 19, 20, 21, 22, 23, 24, 25, 26, 27, 28, 29, 30, 31, 32, 33, 34, or 35 nucleobases of an exon 44, exon 45, exon 50, exon 51, exon 52, or exon 53 target region of the dystrophin pre-mRNA.

Antisense oligonucleotides conjugates of the disclosure target dystrophin pre-mRNA and induce skipping of the targeted exon, so it is excluded or skipped from the mature, spliced mRNA transcript. By skipping the targeted exon, the disrupted reading frame is restored to an in-frame mutation.

The nucleobase sequence of an antisense oligonucleotide conjugate that induces skipping of the targeted exon is designed to be complementary to a specific target sequence within dystrophin pre-mRNA. The antisense oligonucleotide conjugate is a PPMO wherein each morpholino ring of the PMO is linked to a nucleobase including, for example, nucleobases found in DNA (adenine, cytosine, guanine, and thymine) and a cell penetrating peptide.

Exemplary aspects of the disclosure relate to phosphorodiamidate morpholino oligonucleotides of the following general structure:

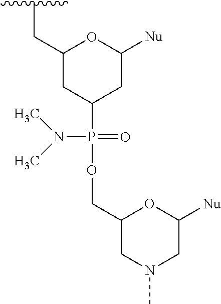

and as described in FIG. 2 of Summerton, J., et al., Antisense & Nucleic Acid Drug Development, 7: 187-195 (1997). Morpholinos as described herein are intended to cover all stereoisomers and tautomers of the foregoing general structure. The synthesis, structures, and binding characteristics of morpholino oligonucleotides are detailed in U.S. Pat. Nos. 5,698,685; 5,217,866; 5,142,047; 5,034,506; 5,166,315; 5,521,063; 5,506,337; 8,076,476; and 8,299,206, all of which are incorporated herein by reference.

In certain aspects, a morpholino is conjugated at the 5′ end of the oligonucleotide with a “tail” moiety to increase its stability and/or solubility. Exemplary tails include:

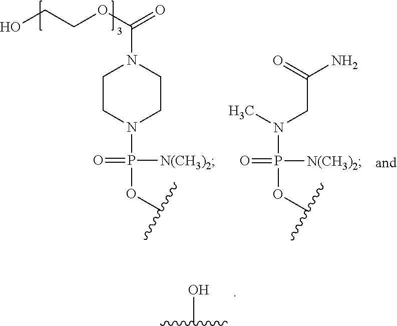

In some aspects, the antisense oligonucleotide conjugate in the composition is of Formula (I):

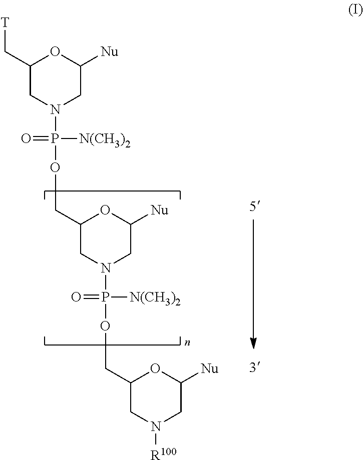

or a pharmaceutically acceptable salt thereof, wherein:
each Nu is a nucleobase which taken together form a targeting sequence;
T is a moiety selected from:

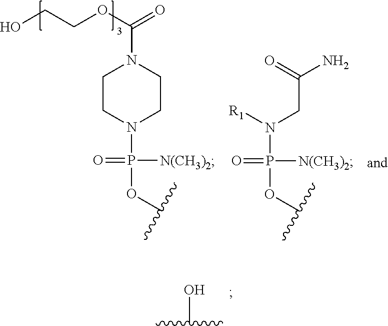

R¹⁰⁰ is a cell penetrating peptide;
each Nu from 1 to m and 5′ to 3′ corresponds to the nucleobases in one of the following:

| | | SEQ ID |
|---|---|---|
| Annealing Site | Base Sequence [5′ to 3′] | NO. |
| H51A(+66+95) | CTC CAA CAT CAA GGA AGA TGG CAT TTC TAG | 1 |
| H51A(+74+97) | ACC TCC AAC ATC AAG GAA GAT GGC | 2 |
| H51A(+70+99) | GTA CCT CCA ACA TCA AGG AAG ATG GCA TTT | 3 |
| H51A(+72+99) | GTA CCT CCA ACA TCA AGG AAG ATG GCA T | 4 |
| H51A(+68+87) | TCA AGG AAG ATG GCA TTT CT | 5 |
| H51A(+68+87) | UCA AGG AmAGm AmUGm GmCA UUU CU | 6 |
| H53A(+36+60) | GTT GCC TCC GGT TCT GAA GGT GTT C | 7 |
| H53A(+36+60) | GTT G5mC5mC T5mC5mC GGT T5mC T GAA GGT GTT 5mC | 8 |
| H53A(+36+56) | CCT CCG GTT CTG AAG GTG TTC | 9 |
| H53A(+23+47) | CTG AAG GTG TTC TTG TAC TTC ATC C | 10 |
| H53A(+32+56) | CCT CCG GTT CTG AAG GTG TTC TTG T | 11 |
| H53A(+33+60) | GTT GCC TCC GGT TCT GAA GGT GTT CTT G | 12 |
| H53A(+30+59) | TTG CCT CCG GTT CTG AAG GTG TTC TTG TAC | 13 |
| H53A(+39+62) | CTG TTG CCT CCG GTT CTG AAG GTG | 14 |
| H53A(+36+69) | CAT TCA ACT GTT GCC TCC GGT TCT GAA GGT G | 15 |
| H53A(+45+62) | CTG TTG CCT CCG GTT CTG | 16 |
| H45A(−03+19) | CAA TGC CAT CCT GGA GTT CCT G | 17 |
| H45A(−09+25) | GCT GCC CAA TGC CAT CCT GGA GTT CCT GTA AGA T | 18 |
| H45A(−03+25) | GCT GCC CAA TGC CAT CCT GGA GTT CCT G | 19 |
| H45A(−06+25) | GCT GCC CAA TGC CAT CCT GGA GTT CCT GTA A | 20 |
| H45A(−12+19) | CAA TGC CAT CCT GGA GTT CCT GTA AGA TAC C | 21 |
| H45A(−09+19) | CAA TGC CAT CCT GGA GTT CCT GTA AGA T | 22 |
| H45A(−12+16) | TGC CAT CCT GGA GTT CCT GTA AGA TAC C | 23 |
| H45A(−14+25) | GCT GCC CAA TGC CAT CCT GGA GTT CCT GTA AGA TAC | 24 |
| | CAA | |
| H45A(−08+19) | CAA TGC CAT CCT GGA GTT CCT GTA AGA | 25 |
| HM45A(−07+25) | GCT GCC CAA TGC CAT CCT GGA GTT CCT GTA AG | 26 |
| H45A(−12+22) | GCC CAA TGC CAT CCT GGA GTT CCT GTA AGA TAC C | 27 |
| H45A(−09+22) | GCC CAA TGC CAT CCT GGA GTT CCT GTA AGA T | 28 |
| H45A(−09+30) | TTG CCG CTG CCC AAT GCC ATC CTG GAG TTC CTG TAA | 29 |
| | GAT | |
| H45A(−06+22) | GCC CAA TGC CAT CCT GGA GTT CCT GTA A | 30 |
| H45A(−06+28) | GCC GCT GCC CAA TGC CAT CCT GGA GTT CCT GTA A | 31 |
| H45A(−03+22) | GCC CAA TGC CAT CCT GGA GTT CCT G | 32 |
| H45A(−03+28) | GCC GCT GCC CAA TGC CAT CCT GGA GTT CCT G | 33 |
| H45A(+9+26) | m5C-G-m5C-T-G-C-m5C-m5C-A-A-T-G-m5C-m5C-A-U-m5C- | 34 |
| | m5C | |
| H44A(−10+15) | GAT CTG TCA AAT CGC CTG CAG GTA A | 35 |
| H44A(−07+15) | GAT CTG TCA AAT CGC CTG CAG G | 36 |
| H44M(−07+17) | CAG ATC TGT CAA ATC GCC TGC AGG | 37 |
| H44A(−08+15) | GAT CTG TCA AAT CGC CTG CAG GT | 38 |
| H44A(−06+15) | GAT CTG TCA AAT CGC CTG CAG | 39 |
| H44A(−08+17) | CAG ATC TGT CAA ATC GCC TGC AGG T | 40 |
| H44A(−06+17) | CAG ATC TGT CAA ATC GCC TGC AG | 41 |
| H50D(+04−18) | GGG ATC CAG TAT ACT TAC AGG C | 42 |
| H50D(+07−18) | GGG ATC CAG TAT ACT TAC AGG CTC C | 43 |
| H50D(+07−16) | GAT CCA GTA TAC TTA CAG GCT CC | 44 |
| H50D(+07−17) | GGA TCC AGT ATA CTT ACA GGC TCC | 45 |
| H50A(−19+07) | ACT TCC TCT TTA ACA GAA AAG CAT AC | 46 |
| H50D(+07−15) | ATC CAG TAT ACT TAC AGG CTC C | 47 |
| H50A(−02+23) | GAG CTC AGA TCT TCT AAC TTC CTC T | 48 |
| H50D(+06−18) | GGG ATC CAG TAT ACT TAC AGG CTC | 49 |
| H50D(+07−20) | ATG GGA TCC AGT ATA CTT ACA GGC TCC | 50 |
| H52A(−01+24) | CTG TTC CAA ATC CTG CAT TGT TGC C | 51 |

wherein A is

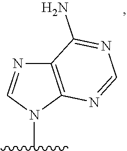 [SMILES: *n1cnc2c(N)ncnc21]

### C is

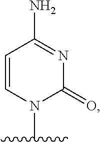 [SMILES: *n1ccc(N)nc1=O]

### G is

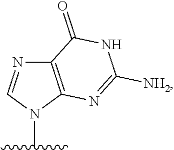 [SMILES: *n1cnc2c(=O)[nH]c(N)nc21]

### T is

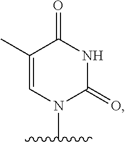 [SMILES: *n1cc(C)c(=O)[nH]c1=O]

### U is

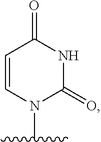 [SMILES: *n1ccc(=O)[nH]c1=O]

Gm is methylated guanine, Am is methylated adenine, and m5C is

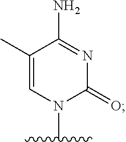 [SMILES: *n1cc(C)c(N)nc1=O]

In some aspects, each Nu from 1 to n and 5′ to 3′ of the antisense oligonucleotide conjugate, or pharmaceutically acceptable salt thereof, in the composition corresponds to SEQ ID NO: 1, SEQ ID NO: 7, SEQ ID NO: 17, or SEQ ID NO: 51. In some aspects, each Nu from 1 to n and 5′ to 3′ of the antisense oligonucleotide conjugate, or pharmaceutically acceptable salt thereof, in the composition corresponds to SEQ ID NO: 1. In some aspects, each Nu from 1 to n and 5′ to 3′ of the antisense oligonucleotide conjugate, or pharmaceutically acceptable salt thereof, in the composition corresponds to SEQ ID NO: 7. In some aspects, each Nu from 1 to n and 5′ to 3′ of the antisense oligonucleotide conjugate, or pharmaceutically acceptable salt thereof, in the composition corresponds to SEQ ID NO: 17. In some aspects, each Nu from 1 to n and 5′ to 3′ of the antisense oligonucleotide conjugate, or pharmaceutically acceptable salt thereof, in the composition corresponds to SEQ ID NO: 51.

In some aspects, T′ in the antisense oligonucleotide conjugate, or pharmaceutically acceptable salt thereof, of Formula (I) in the composition is

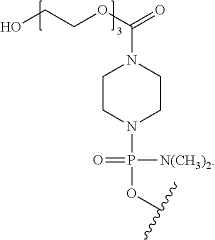

The antisense oligonucleotide conjugate, or pharmaceutically acceptable salt thereof, comprises a cell penetrating peptide that is an arginine-rich peptide. In some aspects, the antisense oligonucleotide conjugate comprises a cell penetrating peptide that is an arginine-rich peptide selected from the group consisting of —(RXR)₄—R^{a} (SEQ ID NO: 54), R-(FFR)₃-R^{a} (SEQ ID NO: 55), —B—X—(RXR)₄—R^{a} (SEQ ID NO: 56), —B—X—R-(FFR)₃-R^{a} (SEQ ID NO: 57), -GLY-R-(FFR)₃-R^{a} (SEQ ID NO: 58), -GLY-R₅—R^{a} (SEQ ID NO: 59), —R₅—R^{a} (SEQ ID NO: 60), -GLY-R₆—R^{a} (SEQ ID NO: 52) and —R₆—R^{a} (SEQ ID NO: 53), wherein R^{a} is selected from H, acyl, benzoyl, and stearoyl, and wherein R is arginine, X is 6-aminohexanoic acid, B is β-alanine, F is phenylalanine and GLY (or G) is glycine. In some aspects, the arginine-rich peptide is -GLY-R₅—R^{a} (SEQ ID NO: 59), —R₅—R^{a} (SEQ ID NO: 60), -GLY-R₆—R^{a} (SEQ ID NO: 52) or —R₆—R^{a} (SEQ ID NO: 53), wherein R is arginine and R^{a} is hydrogen or an acyl group. In some aspects, the arginine-rich peptide.

In some aspects, the antisense oligonucleotide conjugate in the composition is of Formula (II):

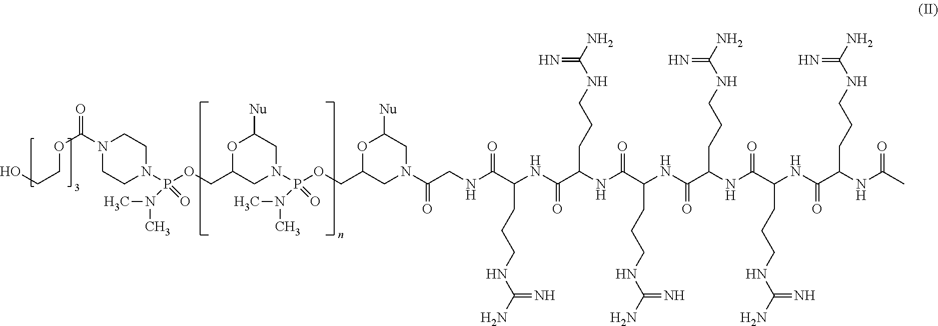

or a pharmaceutically acceptable salt thereof, each Nu from 1 to (n+1) and 5′ to 3′ corresponds to the nucleobases in one of the following:

| | SEQ |
|---|---|
| | ID |
| Base Sequence [5′ to 3′] | NO. |
| CTC CAA CAT CAA GGA AGA TGG CAT TTC TAG | 1 |
| ACC TCC AAC ATC AAG GAA GAT GGC | 2 |
| GTA CCT CCA ACA TCA AGG AAG ATG GCA TTT | 3 |
| GTA CCT CCA ACA TCA AGG AAG ATG GCA T | 4 |
| TCA AGG AAG ATG GCA TTT CT | 5 |
| UCA AGG AmAGm AmUGm GmCA UUU CU | 6 |
| GTT GCC TCC GGT TCT GAA GGT GTT C | 7 |
| GTT G5mC5mC T5mC5mC GGT T5mC T GAA GGT GTT | 8 |
| 5mC | |
| CCT CCG GTT CTG AAG GTG TTC | 9 |
| CTG AAG GTG TTC TTG TAC TTC ATC C | 10 |
| CCT CCG GTT CTG AAG GTG TTC TTG T | 11 |
| GTT GCC TCC GGT TCT GAA GGT GTT CTT G | 12 |
| TTG CCT CCG GTT CTG AAG GTG TTC TTG TAC | 13 |
| CTG TTG CCT CCG GTT CTG AAG GTG | 14 |
| CAT TCA ACT GTT GCC TCC GGT TCT GAA GGT G | 15 |
| CTG TTG CCT CCG GTT CTG | 16 |
| CAA TGC CAT CCT GGA GTT CCT G | 17 |
| GCT GCC CAA TGC CAT CCT GGA GTT CCT GTA AGA | 18 |
| T | |
| GCT GCC CAA TGC CAT CCT GGA GTT CCT G | 19 |
| GCT GCC CAA TGC CAT CCT GGA GTT CCT GTA A | 20 |
| CAA TGC CAT CCT GGA GTT CCT GTA AGA TAC C | 21 |
| CAA TGC CAT CCT GGA GTT CCT GTA AGA T | 22 |
| TGC CAT CCT GGA GTT CCT GTA AGA TAC C | 23 |
| GCT GCC CAA TGC CAT CCT GGA GTT CCT GTA AGA | 24 |
| TAC CAA | |
| CAA TGC CAT CCT GGA GTT CCT GTA AGA | 25 |
| GCT GCC CAA TGC CAT CCT GGA GTT CCT GTA AG | 26 |
| GCC CAA TGC CAT CCT GGA GTT CCT GTA AGA TAC | 27 |
| C | |
| GCC CAA TGC CAT CCT GGA GTT CCT GTA AGA T | 28 |
| TTG CCG CTG CCC AAT GCC ATC CTG GAG TTC CTG | 29 |
| TAA GAT | |
| GCC CAA TGC CAT CCT GGA GTT CCT GTA A | 30 |
| GCC GCT GCC CAA TGC CAT CCT GGA GTT CCT GTA A | 31 |
| GCC CAA TGC CAT CCT GGA GTT CCT G | 32 |
| GCC GCT GCC CAA TGC CAT CCT GGA GTT CCT G | 33 |
| m5C-G-m5C-T-G-C-m5C-m5C-A-A-T-G-m5C-m5C-A-U- | 34 |
| m5C-m5C | |
| GAT CTG TCA AAT CGC CTG CAG GTA A | 35 |
| GAT CTG TCA AAT CGC CTG CAG G | 36 |
| CAG ATC TGT CAA ATC GCC TGC AGG | 37 |
| GAT CTG TCA AAT CGC CTG CAG GT | 38 |
| GAT CTG TCA AAT CGC CTG CAG | 39 |
| CAG ATC TGT CAA ATC GCC TGC AGG T | 40 |
| CAG ATC TGT CAA ATC GCC TGC AG | 41 |
| GGG ATC CAG TAT ACT TAC AGG C | 42 |
| GGG ATC CAG TAT ACT TAC AGG CTC C | 43 |
| GAT CCA GTA TAC TTA CAG GCT CC | 44 |
| GGA TCC AGT ATA CTT ACA GGC TCC | 45 |
| ACT TCC TCT TTA ACA GAA AAG CAT AC | 46 |
| ATC CAG TAT ACT TAC AGG CTC C | 47 |
| GAG CTC AGA TCT TCT AAC TTC CTC T | 48 |
| GGG ATC CAG TAT ACT TAC AGG CTC | 49 |
| ATG GGA TCC AGT ATA CTT ACA GGC TCC | 50 |
| CTG TTC CAA ATC CTG CAT TGT TGC C | 51 |

wherein A is

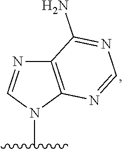 [SMILES: *n1cnc2c(N)ncnc21]

### C is

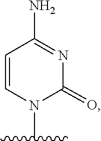 [SMILES: *n1ccc(N)nc1=O]

### G is

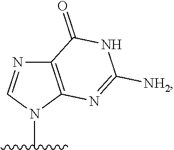 [SMILES: *n1cnc2c(=O)[nH]c(N)nc21]

### T is

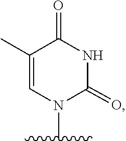 [SMILES: *n1cc(C)c(=O)[nH]c1=O]

### U is

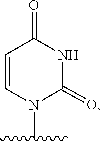 [SMILES: *n1ccc(=O)[nH]c1=O]

Gm is methylated guanine, Am is methylated adenine, and m5C is

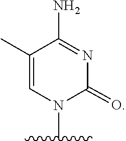 [SMILES: *n1cc(C)c(N)nc1=O]

In some aspects, each Nu from 1 to n and 5′ to 3′ of the antisense oligonucleotide conjugate of Formula (II), or pharmaceutically acceptable salt thereof, in the composition corresponds to SEQ ID NO: 1, SEQ ID NO: 7, SEQ ID NO: 17, or SEQ ID NO: 51. In some aspects, each Nu from 1 to n and 5′ to 3′ of the antisense oligonucleotide conjugate of Formula (II), or pharmaceutically acceptable salt thereof, in the composition corresponds to SEQ ID NO: 1. In some aspects, each Nu from 1 to n and 5′ to 3′ of the antisense oligonucleotide conjugate of Formula (II), or pharmaceutically acceptable salt thereof, in the composition corresponds to SEQ ID NO: 7. In some aspects, each Nu from 1 to n and 5′ to 3′ of the antisense oligonucleotide conjugate of Formula (II), or pharmaceutically acceptable salt thereof, in the composition corresponds to SEQ ID NO: 17. In some aspects, each Nu from 1 to n and 5′ to 3′ of the antisense oligonucleotide conjugate of Formula (II), or pharmaceutically acceptable salt thereof, in the composition corresponds to SEQ ID NO: 51.

In some aspects, the antisense oligonucleotide conjugate in the composition is of Formula (III):

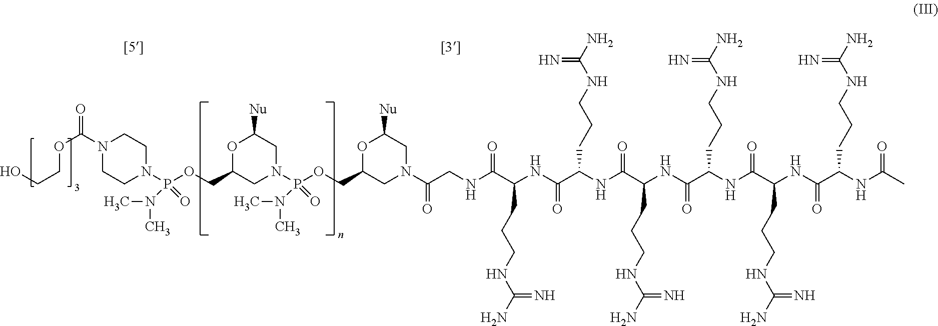

or a pharmaceutically acceptable salt thereof, each Nu from 1 to (n+1) and 5′ to 3′ corresponds to the nucleobases in one of the following:

| | SEQ |
|---|---|
| | ID |
| Base Sequence [5′ to 3′] | NO. |
| CTC CAA CAT CAA GGA AGA TGG CAT TTC TAG | 1 |
| ACC TCC AAC ATC AAG GAA GAT GGC | 2 |
| GTA CCT CCA ACA TCA AGG AAG ATG GCA TTT | 3 |
| GTA CCT CCA ACA TCA AGG AAG ATG GCA T | 4 |
| TCA AGG AAG ATG GCA TTT CT | 5 |
| UCA AGG AmAGm AmUGm GmCA UUU CU | 6 |
| GTT GCC TCC GGT TCT GAA GGT GTT C | 7 |
| GTT G5mC5mC T5mC5mC GGT T5mC T GAA GGT GTT | 8 |
| 5mC | |
| CCT CCG GTT CTG AAG GTG TTC | 9 |
| CTG AAG GTG TTC TTG TAC TTC ATC C | 10 |
| CCT CCG GTT CTG AAG GTG TTC TTG T | 11 |
| GTT GCC TCC GGT TCT GAA GGT GTT CTT G | 12 |
| TTG CCT CCG GTT CTG AAG GTG TTC TTG TAC | 13 |
| CTG TTG CCT CCG GTT CTG AAG GTG | 14 |
| CAT TCA ACT GTT GCC TCC GGT TCT GAA GGT G | 15 |
| CTG TTG CCT CCG GTT CTG | 16 |
| CAA TGC CAT CCT GGA GTT CCT G | 17 |
| GCT GCC CAA TGC CAT CCT GGA GTT CCT GTA AGA | 18 |
| T | |
| GCT GCC CAA TGC CAT CCT GGA GTT CCT G | 19 |
| GCT GCC CAA TGC CAT CCT GGA GTT CCT GTA A | 20 |
| CAA TGC CAT CCT GGA GTT CCT GTA AGA TAC C | 21 |
| CAA TGC CAT CCT GGA GTT CCT GTA AGA T | 22 |
| TGC CAT CCT GGA GTT CCT GTA AGA TAC C | 23 |
| GCT GCC CAA TGC CAT CCT GGA GTT CCT GTA AGA | 24 |
| TAC CAA | |
| CAA TGC CAT CCT GGA GTT CCT GTA AGA | 25 |
| GCT GCC CAA TGC CAT CCT GGA GTT CCT GTA AG | 26 |
| GCC CAA TGC CAT CCT GGA GTT CCT GTA AGA TAC | 27 |
| C | |
| GCC CAA TGC CAT CCT GGA GTT CCT GTA AGA T | 28 |
| TTG CCG CTG CCC AAT GCC ATC CTG GAG TTC CTG | 29 |
| TAA GAT | |
| GCC CAA TGC CAT CCT GGA GTT CCT GTA A | 30 |
| GCC GCT GCC CAA TGC CAT CCT GGA GTT CCT GTA A | 31 |
| GCC CAA TGC CAT CCT GGA GTT CCT G | 32 |
| GCC GCT GCC CAA TGC CAT CCT GGA GTT CCT G | 33 |
| m5C-G-m5C-T-G-C-m5C-m5C-A-A-T-G-m5C-m5C-A-U- | 34 |
| m5C-m5C | |
| GAT CTG TCA AAT CGC CTG CAG GTA A | 35 |
| GAT CTG TCA AAT CGC CTG CAG G | 36 |
| CAG ATC TGT CAA ATC GCC TGC AGG | 37 |
| GAT CTG TCA AAT CGC CTG CAG GT | 38 |
| GAT CTG TCA AAT CGC CTG CAG | 39 |
| CAG ATC TGT CAA ATC GCC TGC AGG T | 40 |
| CAG ATC TGT CAA ATC GCC TGC AG | 41 |
| GGG ATC CAG TAT ACT TAC AGG C | 42 |
| GGG ATC CAG TAT ACT TAC AGG CTC C | 43 |
| GAT CCA GTA TAC TTA CAG GCT CC | 44 |
| GGA TCC AGT ATA CTT ACA GGC TCC | 45 |
| ACT TCC TCT TTA ACA GAA AAG CAT AC | 46 |
| ATC CAG TAT ACT TAC AGG CTC C | 47 |
| GAG CTC AGA TCT TCT AAC TTC CTC T | 48 |
| GGG ATC CAG TAT ACT TAC AGG CTC | 49 |
| ATG GGA TCC AGT ATA CTT ACA GGC TCC | 50 |
| CTG TTC CAA ATC CTG CAT TGT TGC C | 51 |

wherein A is

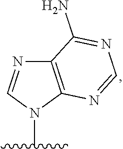 [SMILES: *n1cnc2c(N)ncnc21]

### C is

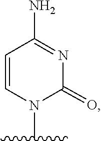 [SMILES: *n1ccc(N)nc1=O]

### G is

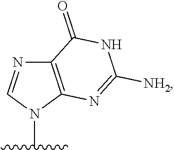 [SMILES: *n1cnc2c(=O)[nH]c(N)nc21]

### T is

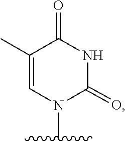 [SMILES: *n1cc(C)c(=O)[nH]c1=O]

### U is

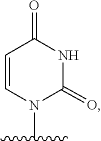 [SMILES: *n1ccc(=O)[nH]c1=O]

Gm is methylated guanine, Am is methylated adenine, and m5C is

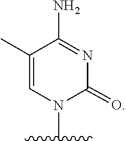 [SMILES: *n1cc(C)c(N)nc1=O]

In some aspects, each Nu from 1 to n and 5′ to 3′ of the antisense oligonucleotide conjugate of Formula (III), or pharmaceutically acceptable salt thereof, in the composition corresponds to SEQ ID NO: 1, SEQ ID NO: 7, SEQ ID NO: 17, or SEQ ID NO: 51. In some aspects, each Nu from 1 to n and 5′ to 3′ of the antisense oligonucleotide conjugate of Formula (III), or pharmaceutically acceptable salt thereof, in the composition corresponds to SEQ ID NO: 1. In some aspects, each Nu from 1 to n and 5′ to 3′ of the antisense oligonucleotide conjugate of Formula (III), or pharmaceutically acceptable salt thereof, in the composition corresponds to SEQ ID NO: 7. In some aspects, each Nu from 1 to n and 5′ to 3′ of the antisense oligonucleotide conjugate of Formula (III), or pharmaceutically acceptable salt thereof, in the composition corresponds to SEQ ID NO: 17. In some aspects, each Nu from 1 to n and 5′ to 3′ of the antisense oligonucleotide conjugate of Formula (III), or pharmaceutically acceptable salt thereof, in the composition corresponds to SEQ ID NO: 51.

In some aspects, the antisense oligonucleotide conjugate in the composition is PPMO #1, PPMO #2, PPMO #3, or PPMO #4, or a pharmaceutically acceptable salt thereof. In some aspects, the antisense oligonucleotide conjugate in the composition is PPMO #1, or a pharmaceutically acceptable salt thereof. In some aspects, the antisense oligonucleotide conjugate in the composition is PPMO #2, or a pharmaceutically acceptable salt thereof. In some aspects, the antisense oligonucleotide conjugate in the composition is PPMO #3, or a pharmaceutically acceptable salt thereof. In some aspects, the antisense oligonucleotide conjugate in the composition is PPMO #4, or a pharmaceutically acceptable salt thereof.

In some aspects, the composition comprises about 50 mg to about 500 mg of the antisense oligonucleotide conjugate, or a pharmaceutically acceptable salt thereof. In some aspects, the composition comprises about 50 mg to about 450 mg of the antisense oligonucleotide conjugate, or a pharmaceutically acceptable salt thereof. In some aspects, the composition comprises about 50 mg to about 400 mg, about 50 mg to about 350 mg, about 50 mg to about 300 mg, about 50 mg to about 250 mg, about 50 mg to about 200 mg, about 50 mg to about 150 mg, about 50 mg to about 100 mg, about 75 mg to about 500 mg, about 75 mg to about 450 mg, about 75 mg to about 400 mg, about 75 mg to about 350 mg, about 75 mg to about 300 mg, about 75 mg to about 250 mg, about 75 mg to about 200 mg, about 75 mg to about 150 mg, about 100 mg to about 500 mg, about 100 mg to about 450 mg, about 100 mg to about 400 mg, about 100 mg to about 350 mg, about 100 mg to about 300 mg, about 100 mg to about 200 mg, about 100 mg to about 150 mg, about 125 mg to about 500 mg, about 125 mg to about 450 mg, about 125 mg to about 400 mg, about 125 mg to about 350 mg, about 125 mg to about 300 mg, about 125 mg to about 250 mg, about 125 mg to about 200 mg, about 150 mg to about 500 mg, about 150 mg to about 450 mg, about 150 mg to about 400 mg, about 150 mg to about 350 mg, about 150 mg to about 300 mg, about 150 mg to about 250 mg, about 150 mg to about 200 mg, about 175 mg to about 500 mg, about 175 mg to about 450 mg, about 175 mg to about 400 mg, about 175 mg to about 350 mg, about 175 mg to about 300 mg, about 175 mg to about 250 mg, about 200 mg to about 500 mg, about 200 mg to about 450 mg, about 200 mg to about 400 mg, about 200 mg to about 350 mg, about 200 mg to about 300 mg, about 200 mg to about 250 mg, about 225 mg to about 500 mg, about 225 mg to about 450 mg, about 225 mg to about 400 mg, about 225 mg to about 350 mg, about 225 mg to about 300 mg, about 250 mg to about 500 mg, about 250 mg to about 450 mg, about 250 mg to about 400 mg, about 250 mg to about 350 mg, about 275 mg to about 500 mg, about 275 mg to about 450 mg, about 275 mg to about 400 mg, about 275 mg to about 350 mg, about 300 mg to about 500 mg, about 300 mg to about 450 mg, about 300 mg to about 400 mg, about 300 mg to about 350 mg, about 325 mg to about 500 mg, about 325 mg to about 450 mg, about 325 mg to about 400 mg, about 350 mg to about 500 mg, about 350 mg to about 450 mg, about 350 mg to about 400 mg, about 400 mg to about 500 mg, about 400 mg to about 450 mg, or about 450 mg to about 500 mg of the antisense oligonucleotide conjugate, or a pharmaceutically acceptable salt thereof. In some aspects, the composition comprises about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg of the antisense oligonucleotide conjugate based upon the weight of the free base. The weight of the antisense oligonucleotide conjugate as its particular pharmaceutically acceptable salt to be included in the composition would be adjusted. For example, 102 mg PPMO #1 as its hexachloride (6HCl) salt form is equivalent to 100 mg of PPMO #1 as its free base.

### B. Pharmaceutically Acceptable Salts of Antisense Oligonucleotide Conjugates

Certain aspects of antisense oligonucleotides described herein can contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of antisense oligonucleotides of the present disclosure. These salts can be prepared in situ in the administration vehicle or the dosage form manufacturing process, or by separately reacting a purified antisense oligonucleotide of the disclosure in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, tosylate, citrate, maleate, fumarate, succinate, tartrate, naphthylate, mesylate, glucoheptonate, lactobionate, and laurylsulphonate salts and the like. (See, e.g., Berge et al. (1977) “Pharmaceutical Salts”, J. Pharm. Sci. 66:1-19).

The pharmaceutically acceptable salts of the subject antisense oligonucleotides include the conventional nontoxic salts or quaternary ammonium salts of the antisense oligonucleotides, e.g., from non-toxic organic or inorganic acids. For example, such conventional nontoxic salts include those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In certain aspects, the antisense oligonucleotides of the present disclosure can contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of antisense oligonucleotides of the present disclosure. These salts can likewise be prepared in situ in the administration vehicle or the dosage form manufacturing process, or by separately reacting the purified antisense oligonucleotide in its free acid form with a suitable base, such as the hydroxide, carbonate, or bicarbonate of a pharmaceutically-acceptable metal cation, with ammonia, or with a pharmaceutically-acceptable organic primary, secondary, or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, e.g., Berge et al., supra).

The salt form can be a complex of multiple cations or anions with the antisense oligonucleotide conjugate. For example, the salt form can be a monohalide (1HCl), dihalide (2HCl), trihalide (3HCl), tetrahalide (4HCl), pentahalide (5HCl), or hexahalide (6HCl).

### B. Surfactants

The compositions of the disclosure comprise one or more surfactants. In some aspects, the one or more surfactants are present in the composition in an amount between about 0.001% and about 2% by weight of the composition. In some aspects, the one or more surfactants are present in the composition in an amount between about 0.002% and about 1% by weight of the composition. In some aspects, the one or more surfactants are present in the composition in an amount between about 0.01% and about 1% by weight of the composition.

In some aspects, the one or more surfactants in the composition is selected from the group consisting of polyoxyethylene sorbitan monooleate (e.g., polysorbate 80, polysorbate 20), polyvinylpyrrolidone (PVP, povidone, poloxamer, poloxamer F188), and mixtures thereof. In some aspects, the surfactant is polyoxyethylene sorbitan monooleate (e.g., polysorbate 80, polysorbate 20). In some aspects, the surfactant is polyvinylpyrrolidone.

In some aspects, the composition contains polyoxyethylene sorbitan monooloeate (e.g., polysorbate 80, polysorbate 20) in an amount between about 0.001% and about 2% by weight of the composition. In some aspects, the composition contains polyoxyetheylene sorbitan monooleate (e.g., polysorbate 80, polysorbate 20) in an amount between about 0.001% and about 2% by weight of the composition. In some aspects, the composition contains polyoxyethylene sorbitan monooleate (e.g., polysorbate 80, polysorbate 20) in an amount between about 0.002% and about 1% by weight of the composition. In some aspects, the composition contains polyoxyethylene sorbitan monooleate (e.g., polysorbate 80, polysorbate 20) in an amount between about 0.01% and about 1% by weight of the composition.

In some aspects, the composition contains polyvinylpyrrolidone in an amount between about 0.001% and about 2% by weight of the composition. In some aspects, the composition contains polyvinylpyrrolidone in an amount between about 0.001% and about 2% by weight of the composition. In some aspects, the composition contains polyvinylpyrrolidone in an amount between about 0.002% and about 1% by weight of the composition. In some aspects, the composition contains in an amount between about 0.01% and about 1% by weight of the composition.

### C. Sugars

The compositions of the disclosure comprise one or more sugars. In some aspects, the one or more sugars are present in the composition in an amount between about 0.1% and about 11% by weight of the composition. In some aspects, the one or more sugars are present in the composition in an amount between about 0.5% and about 7% by weight of the composition. In some aspects, the one or more sugars are present in the composition in an amount of up to 6% by weight of the composition. In some aspects, the one or more sugars are present in the composition in an amount of about 6% by weight of the composition.

In some aspects, the one or more sugars are selected from the group consisting of trehalose, mannitol, sucrose, and mixtures thereof. In some aspects, the sugar is a mixture of mannitol and sucrose. In some aspects, the sugar is mannitol. In some aspects, the sugar is sucrose.

In some aspects, the composition contains a mixture of mannitol and sucrose in an amount between about 0.1% and about 11% by weight of the composition. In some aspects, the composition contains a mixture of mannitol and sucrose in an amount between about 0.5% and about 7% by weight of the composition. In some aspects, the composition contains a mixture of mannitol and sucrose in an amount of up to 6% by weight of the composition. In some aspects, the one or more sugars are present in the composition in an amount of about 6% by weight of the composition.

In some aspects, the sugar is mannitol and is present in the composition in an amount between about 0.1% and about 11% by weight of the composition. In some aspects, the sugar is mannitol and is present in the composition in an amount between about 0.5% and about 7% by weight of the composition. In some aspects, the sugar is mannitol and is present in the composition in an amount of up to 6% by weight of the composition. In some aspects, the sugar is mannitol and is present in the composition in an amount of about 6% by weight of the composition.

In some aspects, the sugar is sucrose and is present in the composition in an amount between about 0.1% and about 11% by weight of the composition. In some aspects, the sugar is sucrose and is present in the composition in an amount between about 0.5% and about 7% by weight of the composition. In some aspects, the sugar is sucrose and is present in the composition in an amount of up to 6% by weight of the composition. In some aspects, the sugar is sucrose and is present in the composition in an amount of about 6% by weight of the composition.

### D. Buffering Agent

The compositions of the disclosure comprise one or more buffering agents. In some aspects, the one or more buffering agents are present in an amount sufficient to provide a pH of about 6.0 to about 7.0 when the composition is dissolved or dispersed in an aqueous solvent. In some aspects, the one or more buffering agents are present in an amount sufficient to provide a pH of about 6.3 to about 6.5. In some aspects, the one or more buffering agents are present in an amount sufficient to provide a pH of about 6.0, about 6.1, about 6.2, about 6.3, about 6.4, about 6.5, about 6.6, about 6.7, about 6.8, about 6.9, or about 7.0. In some aspects, the one or more buffering agents are present in an amount sufficient to provide a pH of about 6.3. In some aspects, the one or more buffering agents are present in an amount sufficient to provide a pH of about 6.4. In some aspects, the one or more buffering agents are present in an amount sufficient to provide a pH of about 6.5.

In some aspects, the one or more buffering agents are present in a range of about 10 mM to about 50 mM when the composition is dissolved or dispersed in an aqueous solvent. In some aspects, the one or more buffering agents are present in a range of about 10 mM to about 40 mM when the composition is dissolved or dispersed in an aqueous solvent. In some aspects, the one or more buffering agents are present in a range of about 10 mM to about 30 mM when the composition is dissolved or dispersed in an aqueous solvent. In some aspects, the one or more buffering agents are present in a range of about 20 mM to about 50 mM when the composition is dissolved or dispersed in an aqueous solvent. In some aspects, the one or more buffering agents are present in a range of about 20 mM to about 40 mM when the composition is dissolved or dispersed in an aqueous solvent. In some aspects, the one or more buffering agents are present at about 10 mM, about 15 mM, about 20 mM, about 25 mM, about 30 mM, about 35 mM, about 40 mM, about 45 mM, or about 50 mM when the composition is dissolved or dispersed in an aqueous solvent.

In some aspects, the one or more buffering agents are selected from the group consisting of histidine, citrate, maleate, arginine, and mixtures thereof. In some aspects, the buffering agent is citrate.

In some aspects, the composition has an osmolality between about 250 mOsm/kg and about 650 mOsm/kg. In some aspects, the composition has an osmolality between about 300 mOsm/kg and about 500 mOsm/kg. In some aspects, the composition has an osmolality between about 300 mOsm/kg and about 600 mOsm/kg. In some aspects, the composition has an osmolality between about 400 mOsm/kg and about 600 mOsm/kg.

In some aspects, the composition is in lyophilized form. In some aspects, the composition dissolves within 30 minutes after addition of an aqueous solvent. In some aspects, the composition dissolves within 20 minutes after addition of an aqueous solvent. In some aspects, the composition dissolves within 15 minutes after addition of an aqueous solvent. In some aspects, the composition dissolves within 10 minutes after addition of an aqueous solvent. In some aspects, the composition dissolves within 9 minutes, about 8 minutes, about 7 minutes, about 6 minutes, about 5 minutes, about 4 minutes, or about 3 minutes after addition of an aqueous solvent.

In some aspects, the aqueous solvent used to dissolve the composition is sterile water.

### III. Methods of Use

### Restoration of the Dystrophin Reading Frame Using Exon Skipping

A potential therapeutic approach to the treatment of DMD caused by out-of-frame mutations in the dystrophin gene is suggested by the milder form of dystrophinopathy known as BMD, which is caused by in-frame mutations. The ability to convert an out-of-frame mutation to an in-frame mutation would hypothetically preserve the mRNA reading frame and produce an internally shortened yet functional dystrophin protein. Antisense oligonucleotides of the disclosure were designed to accomplish this.

Hybridization of the antisense oligonucleotide conjugate (e.g., antisense oligonucleotide conjugate of Formula (I), Formula (II), or Formula (III)) with the targeted pre-mRNA sequence interferes with formation of the pre-mRNA splicing complex and deletes exon 50 from the mature mRNA. The structure and conformation of antisense oligonucleotides of the disclosure allow for sequence-specific base pairing to the complementary sequence.

Normal dystrophin mRNA containing all 79 exons will produce normal dystrophin protein. The shape of each exon depicts how codons are split between exons; of note, one codon consists of three nucleotides. Rectangular shaped exons start and end with complete codons. Arrow shaped exons start with a complete codon but end with a split codon, containing only nucleotide #1 of the codon. Nucleotides #2 and #3 of this codon are contained in the subsequent exon which will start with a chevron shape.

Clinical outcomes for analyzing the effect of an antisense oligonucleotide conjugate that is complementary to a target region of exon 44, exon 45, exon 50, exon 51, exon 52, or exon 53 of the human dystrophin pre-mRNA and induces exon skipping include percent dystrophin positive fibers (PDPF), six-minute walk test (6MWT), loss of ambulation (LOA), North Star Ambulatory Assessment (NSAA), pulmonary function tests (PFT), ability to rise (from a supine position) without external support, de novo dystrophin production, and other functional measures.

In some aspects, the present disclosure provides methods for producing dystrophin in a subject having a mutation of the dystrophin gene that is amenable to exon 44, exon 45, exon 50, exon 51, exon 52, or exon 53 skipping, the method comprising administering to the subject a composition described herein. In certain aspects, the present disclosure provides methods for restoring an mRNA reading frame to induce dystrophin protein production in a subject with Duchenne muscular dystrophy (DMD) who has a mutation of the dystrophin gene that is amenable to exon 44, exon 45, exon 50, exon 51, exon 52, or exon 53 skipping by administering a composition described herein. Protein production can be measured by reverse-transcription polymerase chain reaction (RT-PCR), western blot analysis, or immunohistochemistry (IHC).

In some aspects, the present disclosure provides methods for treating DMD in a subject in need thereof, wherein the subject has a mutation of the dystrophin gene that is amenable to exon 44, exon 45, exon 50, exon 51, exon 52, or exon 53 skipping, the method comprising administering to the subject a composition described herein. In some aspects, the subject in need thereof is a human patient having DMD.

In various aspects, treatment of the subject is measured by delay of disease progression. In some aspects, treatment of the subject is measured by maintenance of ambulation in the subject or reduction of loss of ambulation in the subject. In some aspects, ambulation is measured using the 6 Minute Walk Test (6MWT). In certain aspects, ambulation is measured using the North Start Ambulatory Assessment (NSAA).

In various aspects, the present disclosure provides methods for maintaining pulmonary function or reducing loss of pulmonary function in a subject with DMD, wherein the subject has a mutation of the DMD gene that is amenable to exon 44, exon 45, exon 50, exon 51, exon 52, or exon 53 skipping, the method comprising administering to the subject a composition described herein. In some aspects, pulmonary function is measured as Maximum Expiratory Pressure (MEP). In certain aspects, pulmonary function is measured as Maximum Inspiratory Pressure (MIP). In some aspects, pulmonary function is measured as Forced Vital Capacity (FVC).

In various aspects, a composition of the disclosure is co-administered with a therapeutically effective amount of a non-steroidal anti-inflammatory compound. In some aspects, the non-steroidal anti-inflammatory compound is an NF-kB inhibitor. For example, in some aspects, the NF-kB inhibitor can be CAT-1004 or a pharmaceutically acceptable salt thereof. In various aspects, the NF-kB inhibitor can be a conjugate of salicylate and DHA. In some aspects, the NF-kB inhibitor is CAT-1041 or a pharmaceutically acceptable salt thereof. In certain aspects, the NF-kB inhibitor is a conjugate of salicylate and EPA. In various aspects, the NF-kB inhibitor is

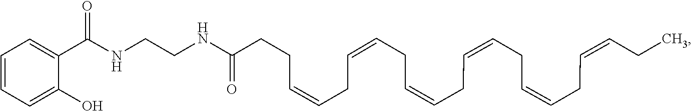 [SMILES: CC/C=C\C/C=C\C/C=C\C/C=C\C/C=C\C/C=C\CCC(=O)NCCNC(=O)c1ccccc1O]

or a pharmaceutically acceptable salt thereof.

In some aspects, the non-steroidal anti-inflammatory compound is a TGF-b inhibitor. For example, in certain aspects, the TGF-b inhibitor is HT-100.

In certain aspects, there is described a composition comprising an antisense oligonucleotide conjugate, or pharmaceutically acceptable salt thereof, as described herein for use in therapy. In certain aspects, there is described a composition comprising an antisense oligonucleotide conjugate, or pharmaceutically acceptable salt thereof, as described herein for use in the treatment of Duchenne muscular dystrophy.

### V. Kits

The disclosure also provides kits for treatment of a patient with a genetic disease which kit comprises at least a composition comprising an antisense oligonucleotide conjugate, or pharmaceutically acceptable salt thereof, packaged in a suitable container, together with instructions for its use. The kits can also contain peripheral reagents such as buffers, stabilizers, etc. Those of ordinary skill in the field should appreciate that applications of the above method has wide application for identifying antisense molecules suitable for use in the treatment of many other diseases. In an aspect, the kit comprises an antisense oligonucleotide conjugate, or pharmaceutically acceptable salt thereof, according to Formula (I), Formula (II), or Formula (III).

### EXAMPLES

Although the foregoing disclosure has been described in some detail by way of illustration and example for purposes of clarity of understanding, it will be readily apparent to one of ordinary skill in the art in light of the teachings of this disclosure that certain changes and modifications can be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### Example 1 (100 mg/Vial Configuration)

### Preparation of PPMO #1 Hexahydrochloride Drug Product

Into 1 L of water for injection, 5.67 g of sodium citrate dihydrate and 0.24 g of citric acid and monohydrate were added. To this citrate buffer solution, 40 g D-Mannitol, 38 g sucrose and 0.02 g of polysorbate 80 were added. 51.0 g PPMO #1 hydrochloride salt (6HCl salt, equivalent to 50.0 g of PPMO #1 free base) was added and mixed well. Upon complete dissolution, solution pH is confirmed as 6.0-7.0, osmolality as 300-500 mOsm/kg and concentration of PPMO #1 is measured using extinction coefficient of 23.1 at 260 nm in 1% ammonium hydroxide. Final solution is filtered through 0.2 μm bioburden reduction filters. 2 mL of this solution is then filled into vials, lyophilized and stoppered and sealed to produce the final drug product vials.

### Reconstitution of PPMO #1 Hexahydrochloride Drug Product

For each PPMO #1 hexahydrochloride drug product vial, 2 mL of sterile water for injection is added using needle/syringe through the stopper without seal/stopper. Contents are mixed of each vial by gently swirling without any shaking. Each vial is visually inspected to confirm the content of each vial is completely dissolved. Gentle inversion of 5-10 times is suggested to ensure homogeneity. Upon reconstitution, PPMO #1 hexahydrochloride is a clear, colorless solution, which may have some opalescence. Reconstituted drug product is then drawn from individual vials and diluted with 0.9% sodium chloride injection (saline) to a concentration of about 20 mg/mL for infusion.

### Analysis

Samples were analyzed for appearance, water content, reconstitution time, and subvisible particle analysis. Appearance of lyophilized cake and reconstitution solution was performed visually for clarity and presence of any visible particles. The moisture contents in the freeze-dried cakes were determined by Coulometric Karl Fisher titration and was found to be less than 1% based on duplicate measurement. The reconstitution time was determined by dissolving the vial contents in 2 mL of water for injection. Dissolution was achieved by gently swirling (not shaking) and inversion of the vials following the addition of the water for injection and was found to be within 5 min. Subvisible particle analysis was performed using light obscuration method USP <788> Method 1. Low volume (1 mL) method was used for this analysis and all analysis met USP criteria of <6000 particle/container for >10 μm and <600 particles/container for >25 μm.

### Example 2 (400 mg/Vial Configuration)

### Preparation of PPMO #1 Hexahydrochloride Drug Product

Into 1 L of water for injection, 5.67 g of sodium citrate dihydrate and 0.24 g of citric acid and monohydrate are added. To this citrate buffer solution, 40 g D-Mannitol, 38 g sucrose and 0.02 g of polysorbate 80 were added. 102.0 g PPMO #1 hexahydrochloride salt (6HCl salt, equivalent to 100.0 g of PPMO #1 free base) was added and mixed well. Upon complete dissolution, solution pH is confirmed as 6.0-7.0, osmolality as 300-500 mOsm/kg and concentration of PPMO #1 is measured using extinction coefficient of 23.1 at 260 nm in 1% ammonium hydroxide. Final solution was filtered through 0.2 μm bioburden reduction filters. 4 mL of this solution was filled into vials, lyophilized, stoppered, and sealed to produce the final drug product vials.

### Reconstitution of PPMO #1 Hexahydrochloride Drug Product

For each PPMO #1 hexahydrochloride drug product vial, 4 mL of sterile water for injection was added using needle/syringe through the stopper without seal/stopper. Contents were mixed of each vial by gently swirling without any shaking. Each vial was visually inspected to confirm the content of each vial is completely dissolved. Gentle inversion of 5-10 times was suggested to ensure homogeneity. Upon reconstitution, PPMO #1 hexahydrochloride was a clear, colorless solution, which may have some opalescence. Reconstituted drug product was then drawn from individual vials and diluted with 0.9% sodium chloride injection (saline) for infusion.

### Analysis

Samples were analyzed for appearance, water content, reconstitution time, and subvisible particle analysis. Appearance of lyophilized cake and reconstitution solution was performed visually for clarity and presence of any visible particles. The moisture contents in the freeze-dried cakes were determined by Coulometric Karl Fisher titration and was found to be less than 1% based on duplicate measurement. The reconstitution time was determined by dissolving the vial contents in 4 mL of water for injection. Dissolution was achieved by gently swirling (not shaking) and inversion of the vials following the addition of the water for injection and was found to be within 15 min. Subvisible particle analysis was performed using light obscuration method USP <788> Method 1. Low volume (1 mL) method was used for this analysis and all analysis met USP criteria of <6000 particle/container for >10 μm and <600 particles/container for >25 μm.

### Example 3 (100 mg/Vial, Polysorbate 80 Variation)

### Preparation of PPMO #1 Hexahydrochloride Drug Product With Varying Amounts of Polysorbate 80

Into 1 L of water for injection, 5.67 g of sodium citrate dihydrate and 0.24 g of citric acid and monohydrate were added. To this citrate buffer solution, 40 g D-Mannitol (about 4% by weight) and 38 g sucrose (about 3.8% by weight) were added. To this solution, four different levels of polysorbate 80, 0 g (no polysorbate 80), 0.002 g (about 0.0002% by weight), 0.02 g (about 0.002% by weight) and 0.2 g (about 0.02% by weight) of polysorbate 80 were added to prepare four different solutions. 51.0 g PPMO #1 hexahydrochloride salt (6HCl salt, equivalent to 50.0 g of PPMO #1 free base) was added to individual solutions and mixed well. Final components of individual solutions are shown in Table 2. Upon complete dissolution, solution pH was confirmed as 6.0-7.0, osmolality as 400-500 mOsm/kg and concentration of PPMO #1 was measured using extinction coefficient of 23.1 at 260 nm in 1% ammonium hydroxide. Final solution was filtered through 0.2 μm bioburden reduction filters. 2 mL of this solution was filled into vials, lyophilized, stoppered, and sealed to produce the final drug product vials.

TABLE 2
Composition of Solutions in Example 3

| Content | Sublot A | Sublot B | Sublot C | Sublot D |
|---|---|---|---|---|
| 20 mM | 20 mM | 20 mM | 20 mM | 20 mM |
| Citrate | Citrate | Citrate | Citrate | Citrate |
| Buffer | Buffer | Buffer | Buffer | Buffer |
| D-Mannitol | 40.0 | 40.0 | 40.0 | 40.0 |
| (mg/mL) | | | | |
| Sucrose | 38.0 | 38.0 | 38.0 | 38.0 |
| (mg/mL) | | | | |
| PPMO#1 | 50.0 | 50.0 | 50.0 | 50.0 |
| (mg/mL) | | | | |
| Polysorbate 80 | 0 | 0.002 | 0.02 | 0.2 |
| (mg/mL) | | | | |
| pH | 6.4 | 6.4 | 6.4 | 6.4 |

### Reconstitution of PPMO #1 Hexahydrochloride Drug Product

For each PPMO #1 hexahydrochloride drug product vial from Example 3, 4 mL of sterile water for injection was added using needle/syringe through the stopper without seal/stopper. Contents were mixed of each vial by gently swirling without any shaking. Each vial was visually inspected to confirm the content of each vial is completely dissolved. Gentle inversion of 5-10 times is suggested to ensure homogeneity. Upon reconstitution, PPMO #1 hexahydrochloride was a clear, colorless solution, which may have some opalescence. Reconstituted drug product was then drawn from individual vials and diluted with 0.9% sodium chloride injection (saline) to a concentration of approximately 20 mg/mL for infusion.

### Analysis

Samples from Example 3 were analyzed for appearance, water content, reconstitution time, and subvisible particle analysis. Appearance of lyophilized cake and reconstitution solution was performed visually for clarity and presence of any visible particles. The moisture contents in the freeze-dried cakes were determined by Coulometric Karl Fisher titration. The reconstitution time was determined by dissolving the vial contents in 2 mL of water for injection. Dissolution was achieved by gently swirling (not shaking) and inversion of the vials following the addition of the water for. Subvisible particle analysis was performed using light obscuration method USP <788> Method 1. Low volume (1 mL) method was used for this analysis.

These samples were put on an accelerated stability conditions (40° C., 75% RH) for 8 weeks. While there was no impact on the appearance of lyophilized cake and moisture content, significant difference in reconstitution time and moderate difference in subvisible particles were observed over time. Lower reconstitution time and no formation of subvisible particles over time can be assigned to level of polysorbate present in those solutions. The results from the accelerated stability conditions are shown in Tables 3-7.

TABLE 3
Appearance of Lyophilized Cake

| Sublot | T = 0 | 1 week | 2 weeks | 3 weeks | 4 weeks | 8 weeks |
|---|---|---|---|---|---|---|
| Sublot | white cracked | white | white | white | white | White cracked |
| A | unshrunken | cracked | cracked | cracked | cracked | cake, no |
| | cakes, shiny | cake, slight | cake, slight | cake, very | cake, very | shrinkage |
| | surface | sheen | sheen | slight sheen | slight sheen | |
| Sublot | white cracked | white | white | white | white | White cracked |
| B | unshrunken | cracked | cracked | cracked | cracked | cake, no |
| | cakes, shiny | cake, slight | cake, slight | cake, very | cake, very | shrinkage |
| | surface | sheen | sheen | slight sheen | slight sheen | |
| Sublot | white cracked | white | white | white | white | White, matte, |
| C | unshrunken | cracked | cracked | cracked | cracked | dense cakes, |
| | cakes, matte | cake, matte | cake, matte | cake, matte | cake, matte | no shrinkage |
| | surface | surface | surface | surface | surface | |
| Sublot | white cracked | white | white | white | white | White cracked |
| D | unshrunken | cracked | cracked | cracked | cracked | cake, no |
| | cakes, matte | cake, matte | cake, matte | cake, matte | cake, matte | shrinkage |
| | surface | surface | surface | surface | surface | |

TABLE 4
Appearance of Reconstituted Solution

| Sublot | T = 0 | 1 week | 2 weeks | 3 weeks | 4 weeks | 8 weeks |
|---|---|---|---|---|---|---|
| Sublot A | Clear and | Clear and | Clear and | Clear and | Clear and | Clear and |
| | Colorless | Colorless | Colorless | Colorless | Colorless | Colorless |
| Sublot B | Clear and | Clear and | Clear and | Clear and | Clear and | Clear and |
| | Colorless | Colorless | Colorless | Colorless | Colorless | Colorless |
| Sublot C | Clear and | Clear and | Clear and | Clear and | Clear and | Clear and |
| | Colorless | Colorless | Colorless | Colorless | Colorless | Colorless |
| Sublot D | Clear and | Clear and | Clear and | Clear and | Clear and | Clear and |
| | Colorless | Colorless | Colorless | Colorless | Colorless | Colorless |

TABLE 5
Moisture (in percent value)

| Sublot | T = 0 | 1 week | 2 weeks | 3 weeks | 4 weeks | 8 weeks |
|---|---|---|---|---|---|---|
| Sublot A | 0.705 | 1.056 | 1.177 | 1.232 | 1.223 | 1.445 |
| Sublot B | 0.578 | 0.948 | 1.067 | 1.133 | 1.159 | 1.316 |
| Sublot C | 0.728 | 0.814 | 0.906 | 0.933 | 0.972 | 1.059 |
| Sublot D | 1.120 | 1.291 | 1.416 | 1.549 | 1.634 | 1.602 |

TABLE 6
Reconstitution Time (in seconds)

| Sublot | T = 0 | 1 week | 2 weeks | 3 weeks | 4 weeks | 8 weeks |
|---|---|---|---|---|---|---|
| Sublot A | 122 | 120 | 104 | 115 | 117 | 96 |
| Sublot B | 122 | 123 | 111 | 110 | 94 | 102 |
| Sublot C | 79 | 78 | 85 | 78 | 64 | 73 |
| Sublot D | 64 | 54 | 69 | 62 | 42 | 49 |

TABLE 7
Analysis of >2 μm particles/mL over time

| | | 2 | 4 | 8 |
|---|---|---|---|---|
| Sublot | T = 0 | weeks | weeks | weeks |
| Sublot A | 7185 | 12219 | 19143 | 26963 |
| Sublot B | 5991 | 5446 | 8904 | 9377 |
| Sublot C | 5227 | 3603 | 3110 | 2845 |
| Sublot D | 2303 | 2988 | 2286 | 1477 |

### Example 4 (100 mg/Vial, Sucrose and Mannitol Variation)

### Preparation of PPMO #1 Hexahydrochloride Drug Product With Varying Amounts of Sucrose and D-Mannitol

Into 1 L of water for injection, 5.67 g of sodium citrate dihydrate and 0.24 g of citric acid and monohydrate were added. To this citrate buffer solution, varying amounts of D-Mannitol and sucrose were added to prepare four different solutions. To each solution, 0.02 g of polysorbate 80 (approximately 0.002% by weight) were added. 51.0 g PPMO #1 hexahydrochloride salt (6HCl salt, equivalent to 50.0 g of PPMO #1 free base) was added to individual solutions and mixed well. Final components of individual solutions of Example 4 are shown in Table 8. Upon complete dissolution, solution pH was confirmed as 6.0-7.0 and osmolality as 400-500 mOsm/kg. The concentration of PPMO #1 was measured using extinction coefficient of 23.1 at 260 nm in 1% ammonium hydroxide. Final solution was filtered through 0.2 μm bioburden reduction filters. 2 mL of this solution was then filled into vials, lyophilized, stoppered, and sealed to produce the final drug product vials.

TABLE 8
Composition of Solutions in Example 4

| | Sublot A | Sublot B | Sublot C | Sublot D |
|---|---|---|---|---|
| 20 mM | 20 mM | 20 mM | 20 mM | 20 mM |
| Citrate | Citrate | Citrate | Citrate | Citrate |
| Buffer | Buffer | Buffer | Buffer | Buffer |
| D-Mannitol | N/A | 20.0 | 40.0 | 60.0 |
| (mg/mL) | | | | |
| Sucrose | 113.0 | 75.0 | 38.0 | N/A |
| (mg/mL) | | | | |
| PPMO#1 | 50.0 | 50.0 | 50.0 | 50.0 |
| (mg/mL) | | | | |
| Polysorbate | 0.02 | 0.02 | 0.02 | 0.02 |
| 80 (mg/mL) | | | | |
| pH | 6.4 | 6.4 | 6.4 | 6.4 |

### Reconstitution of PPMO #1 Hexahydrochloride Drug Product of Example 4

For each PPMO #1 hexahydrochloride drug product vial from Example 4, 4 mL of sterile water for injection was added using needle/syringe through the stopper without seal/stopper. Contents of each vial were mixed by gently swirling without any shaking. Each vial was visually inspected to confirm the content of each vial was completely dissolved. Gentle inversion of 5-10 times is suggested to ensure homogeneity. Upon reconstitution, PPMO #1 hexahydrochloride was a clear, colorless solution, which may have some opalescence. Reconstituted drug product was then drawn from individual vials and diluted with 0.9% sodium chloride injection (saline) to a concentration of approximately 20 mg/mL for infusion.

### Analysis

Samples of Example 4 were analyzed for appearance, water content, reconstitution time, and subvisible particle analysis. Appearance of lyophilized cake and reconstitution solution was performed visually for clarity and presence of any visible particles. The moisture contents in the freeze-dried cakes were determined by Coulometric Karl Fisher titration. The reconstitution time was determined by dissolving the vial contents in 2 mL of water for injection. Dissolution was achieved by gently swirling (not shaking) and inversion of the vials following the addition of the water for. Subvisible particle analysis was performed using light obscuration method USP <788> Method 1. Low volume (1 mL) method was used for this analysis.

These samples of Example 4 were put on an accelerated stability conditions (40° C., 75% RH) for 4 weeks. There was no impact on the appearance of lyophilized cake, moisture content, reconstitution time and subvisible particles were observed over time. Moisture data is shown in Table 9. The results from the accelerated stability conditions are shown in Tables 9-15.

TABLE 9
Moisture (in percent value)

| | | | 2 | 3 | 4 |
|---|---|---|---|---|---|
| Sublot | T = 0 | 1 week | weeks | weeks | weeks |
| Sublot | 0.292 | 0.500 | 0.703 | 0.744 | 0.791 |
| A | | | | | |
| Sublot | 0.202 | 0.470 | 0.624 | 0.650 | 0.770 |
| B | | | | | |
| Sublot | 0.406 | 0.695 | 0.826 | 0.900 | 1.008 |
| C | | | | | |
| Sublot | 0.176 | 0.539 | 0.701 | 0.795 | 0.922 |
| D | | | | | |

TABLE 10
Appearance of Lyophilized Cake

| Sublot | T = 0 | 1 week | 2 weeks | 3 weeks | 4 weeks |
|---|---|---|---|---|---|
| Sublot A | white cracked | white | white | white | white |
| | unshrunken | cracked | cracked cake, slight sheen | cracked | cracked |
| | cakes, shiny | cake, slight | | cake, very | cake, very |
| | surface | sheen | | slight sheen | slight sheen |
| Sublot B | white cracked | white | white | white | white |
| | unshrunken | cracked | cracked cake, slight sheen | cracked | cracked |
| | cakes, shiny | cake, slight | | cake, very | cake, very |
| | surface | sheen | | slight sheen | slight sheen |
| Sublot C | white cracked | white | white | white | white |
| | unshrunken | cracked | cracked cake, matte surface | cracked | cracked |
| | cakes, matte | cake, matte | | cake, matte | cake, matte |
| | surface | surface | | surface | surface |
| Sublot D | white cracked | white | white | white | white |
| | unshrunken | cracked | cracked cake, matte surface | cracked | cracked |
| | cakes, matte | cake, matte | | cake, matte | cake, matte |
| | surface | surface | | surface | surface |

TABLE 11
Appearance of Reconstituted Solution

| Sublot | T = 0 | 1 week | 2 weeks | 3 weeks | 4 weeks |
|---|---|---|---|---|---|
| Sublot | Clear and | Clear and | Clear and | Clear and | Clear and |
| A | Colorless | Colorless | Colorless | Colorless | Colorless |
| Sublot | Clear and | Clear and | Clear and | Clear and | Clear and |
| B | Colorless | Colorless | Colorless | Colorless | Colorless |
| Sublot | Clear and | Clear and | Clear and | Clear and | Clear and |
| C | Colorless | Colorless | Colorless | Colorless | Colorless |
| Sublot | Clear and | Clear and | Clear and | Clear and | Clear and |
| D | Colorless | Colorless | Colorless | Colorless | Colorless |

TABLE 12

| Analysis of >2 μm particles/mL over time | | | | | |
|---|---|---|---|---|---|
| | | 1 | 2 | 3 | 4 |
| Sublot | T = 0 | week | weeks | weeks | weeks |
| Sublot A | 1817 | 2547 | 2510 | 2179 | 3061 |
| Sublot B | 2484 | 2054 | 3183 | 3636 | 2796 |
| Sublot C | 2345 | 1471 | 2485 | 3247 | 3221 |
| Sublot D | 2132 | 2943 | 2405 | 4137 | 1985 |

TABLE 13
Reconstitution Time (in seconds)

| | | 1 | 2 | 3 | 4 |
|---|---|---|---|---|---|
| Sublot | T = 0 | week | weeks | weeks | weeks |
| Sublot A | 82 | 120 | 83 | 90 | 88 |
| Sublot B | 96 | 101 | 87 | 89 | 80 |
| Sublot C | 62 | 91 | 78 | 83 | 77 |
| Sublot D | 83 | 61 | 84 | 85 | 87 |

TABLE 14
pH

| | | 1 | 2 | 3 | 4 |
|---|---|---|---|---|---|
| Sublot | T = 0 | week | weeks | weeks | weeks |
| Sublot A | 6.5 | 6.5 | 6.5 | 6.5 | 6.5 |
| Sublot B | 6.5 | 6.5 | 6.5 | 6.5 | 6.5 |
| Sublot C | 6.5 | 6.5 | 6.5 | 6.5 | 6.5 |
| Sublot D | 6.5 | 6.5 | 6.5 | 6.5 | 6.5 |

TABLE 15
Osmolality (in mOsm/kg)

| | | 1 | 2 | 3 | 4 |
|---|---|---|---|---|---|
| Sublot | T = 0 | week | weeks | weeks | weeks |
| Sublot A | 430 | 428 | 439 | 446 | 454 |
| Sublot B | 428 | 418 | 430 | 442 | 441 |
| Sublot C | 420 | 426 | 428 | 432 | 424 |
| Sublot D | 417 | 426 | 431 | 418 | 436 |

1. A composition, comprising:
a. an antisense oligonucleotide conjugate comprising a cell penetrating peptide covalently attached to a nucleic acid analog, or a pharmaceutically acceptable salt thereof, wherein the cell penetrating peptide includes at least two positively charged amino acids, or a pharmaceutically acceptable salt thereof;
b. one or more surfactants;
c. one or more sugars; and
d. one or more buffering agents.

2-3. (canceled)

4. The composition of claim 1, wherein the one or more surfactants are selected from the group consisting of polyoxyethylene sorbitan monooleate (polysorbate 80, polysorbate 20), polyvinylpyrrolidone (PVP, povidone, poloxamer, poloxamer F188), and any mixtures thereof.

5-6. (canceled)

7. The composition of claim 1, wherein the one or more sugars are selected from the group consisting of trehalose, mannitol, sucrose, and any mixtures thereof.

8-9. (canceled)

10. The composition of claim 1, wherein the one or more buffering agents are present in an amount sufficient to provide a pH of about 6.0 to about 7.0 when the composition is dissolved or dispersed in an aqueous solvent.

11. (canceled)

12. The composition of claim 10, wherein the one or more buffering agents are present in a range of about 10 mM to about 50 mM when the composition is dissolved or dispersed in an aqueous solvent.

13. The composition of claim 10, wherein the one or more buffering agents are selected from the group consisting of histidine, citrate, maleate, arginine, and mixtures thereof.

14. (canceled)

15. The composition of claim 1, wherein the composition has an osmolality between about 250 mOsm/kg and about 650 mOsm/kg.

16-17. (canceled)

18. The composition of claim 1, wherein the composition is in lyophilized form.

19. The composition of claim 1, wherein the composition dissolves within 30 minutes after addition of an aqueous solvent.

20-22. (canceled)

23. The composition of claim 1, wherein the composition comprises about 50 mg to about 500 mg of the antisense oligonucleotide conjugate, or pharmaceutically acceptable salt thereof.

24. The composition of claim 1, wherein the antisense oligonucleotide conjugate is capable of binding a selected target to induce exon skipping in the human dystrophin gene.

25. (canceled)

26. The composition of claim 24, wherein the antisense oligonucleotide conjugate comprises a cell penetrating peptide that is an arginine-rich peptide, wherein the arginine-rich peptide is selected from the group consisting of —(RXR)₄—R^{a} (SEQ ID NO: 54), R-(FFR)₃-R^{a} (SEQ ID NO: 55), —B—X—(RXR)₄—R^{a} (SEQ ID NO: 56), —B—X—R-(FFR)₃-R^{a} (SEQ ID NO: 57), -GLY-R-(FFR)₃-R^{a} (SEQ ID NO: 58), -GLY-R₅—R^{a} (SEQ ID NO: 59), —R₅—R^{a} (SEQ ID NO: 60), -GLY-R₆—R^{a} (SEQ ID NO: 52) and —R₆—R^{a} (SEQ ID NO: 53), wherein R^{a} is selected from H, acyl, benzoyl, and stearoyl, and wherein R is arginine, X is 6-aminohexanoic acid, B is β-alanine, F is phenylalanine and GLY (or G) is glycine.

27-28. (canceled)

29. The composition of claim 1, wherein the antisense oligonucleotide conjugate is of Formula (I): 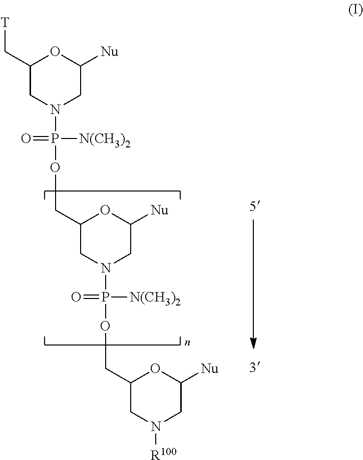
or a pharmaceutically acceptable salt thereof, wherein:
each Nu is a nucleobase which taken together form a targeting sequence;
T is a moiety selected from: 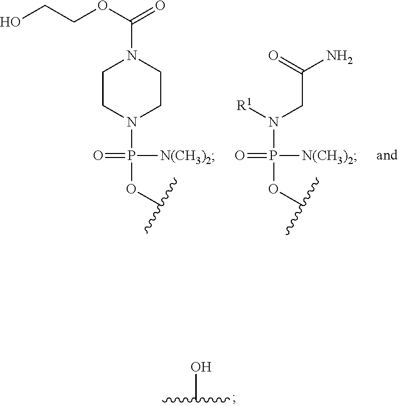
R¹⁰⁰ is the cell penetrating peptide;
each Nu from I to n and 5′ to 3′ corresponds to the nucleobases in one of the following:

| | | SEQ ID |
|---|---|---|
| Annealing Site | Base Sequence [5′ to 3′] | NO. |
| H51A(+66+95) | CTC CAA CAT CAA GGA AGA TGG CAT TTC TAG | 1 |
| H51A(+74+97) | ACC TCC AAC ATC AAG GAA GAT GGC | 2 |
| H51A(+70+99) | GTA CCT CCA ACA TCA AGG AAG ATG GCA TTT | 3 |
| H51A(+72+99) | GTA CCT CCA ACA TCA AGG AAG ATG GCA T | 4 |
| H51A(+68+87) | TCA AGG AAG ATG GCA TTT CT | 5 |
| H51A(+68+87) | UCA AGG AmAGm AmUGm GmCA UUU CU | 6 |
| H53A(+36+60) | GTT GCC TCC GGT TCT GAA GGT GTT C | 7 |
| H53A(+36+60) | GTT G5mC5mC T5mC5mC GGT T5mC T GAA GGT GTT 5mC | 8 |
| H53A(+36+56) | CCT CCG GTT CTG AAG GTG TTC | 9 |
| H53A(+23+47) | CTG AAG GTG TTC TTG TAC TTC ATC C | 10 |
| H53A(+32+56) | CCT CCG GTT CTG AAG GTG TTC TTG T | 11 |
| H53A(+33+60) | GTT GCC TCC GGT TCT GAA GGT GTT CTT G | 12 |
| H53A(+30+59) | TTG CCT CCG GTT CTG AAG GTG TTC TTG TAC | 13 |
| H53A(+39+62) | CTG TTG CCT CCG GTT CTG AAG GTG | 14 |
| H53A(+36+69) | CAT TCA ACT GTT GCC TCC GGT TCT GAA GGT G | 15 |
| H53A(+45+62) | CTG TTG CCT CCG GTT CTG | 16 |
| H45A(−03+19) | CAA TGC CAT CCT GGA GTT CCT G | 17 |
| H45A(−09+25) | GCT GCC CAA TGC CAT CCT GGA GTT CCT GTA AGA T | 18 |
| H45A(−03+25) | GCT GCC CAA TGC CAT CCT GGA GTT CCT G | 19 |
| H45A(−06+25) | GCT GCC CAA TGC CAT CCT GGA GTT CCT GTA A | 20 |
| H45A(−12+19) | CAA TGC CAT CCT GGA GTT CCT GTA AGA TAC C | 21 |
| H45A(−09+19) | CAA TGC CAT CCT GGA GTT CCT GTA AGA T | 22 |
| H45A(−12+16) | TGC CAT CCT GGA GTT CCT GTA AGA TAC C | 23 |
| H45A(−14+25) | GCT GCC CAA TGC CAT CCT GGA GTT CCT GTA AGA | 24 |
| | TAC CAA | |
| H45A(−08+19) | CAA TGC CAT CCT GGA GTT CCT GTA AGA | 25 |
| HM45A(−07+25) | GCT GCC CAA TGC CAT CCT GGA GTT CCT GTA AG | 26 |
| H45A(−12+22) | GCC CAA TGC CAT CCT GGA GTT CCT GTA AGA TAC C | 27 |
| H45A(−09+22) | GCC CAA TGC CAT CCT GGA GTT CCT GTA AGA T | 28 |
| H45A(−09+30) | TTG CCG CTG CCC AAT GCC ATC CTG GAG TTC CTG TAA | 29 |
| | GAT | |
| H45A(−06+22) | GCC CAA TGC CAT CCT GGA GTT CCT GTA A | 30 |
| H45A(−06+28) | GCC GCT GCC CAA TGC CAT CCT GGA GTT CCT GTA A | 31 |
| H45A(−03+22) | GCC CAA TGC CAT CCT GGA GTT CCT G | 32 |
| H45A(−03+28) | GCC GCT GCC CAA TGC CAT CCT GGA GTT CCT G | 33 |
| H45A(+9+26) | m5C-G-m5C-T-G-C-m5C-m5C-A-A-T-G-m5C-m5C-A-U-m5C- | 34 |
| | m5C | |
| H44A(−10+15) | GAT CTG TCA AAT CGC CTG CAG GTA A | 35 |
| H44A(−07+15) | GAT CTG TCA AAT CGC CTG CAG G | 36 |
| H44M(−07+17) | CAG ATC TGT CAA ATC GCC TGC AGG | 37 |
| H44A(−08+15) | GAT CTG TCA AAT CGC CTG CAG GT | 38 |
| H44A(−06+15) | GAT CTG TCA AAT CGC CTG CAG | 39 |
| H44A(−08+17) | CAG ATC TGT CAA ATC GCC TGC AGG T | 40 |
| H44A(−06+17) | CAG ATC TGT CAA ATC GCC TGC AG | 41 |
| H50D(+04−18) | GGG ATC CAG TAT ACT TAC AGG C | 42 |
| H50D(+07−18) | GGG ATC CAG TAT ACT TAC AGG CTC C | 43 |
| H50D(+07−16) | GAT CCA GTA TAC TTA CAG GCT CC | 44 |
| H50D(+07−17) | GGA TCC AGT ATA CTT ACA GGC TCC | 45 |
| H50A(−19+07) | ACT TCC TCT TTA ACA GAA AAG CAT AC | 46 |
| H50D(+07−15) | ATC CAG TAT ACT TAC AGG CTC C | 47 |
| H50A(−02+23) | GAG CTC AGA TCT TCT AAC TTC CTC T | 48 |
| H50D(+06−18) | GGG ATC CAG TAT ACT TAC AGG CTC | 49 |
| H50D(+07−20) | ATG GGA TCC AGT ATA CTT ACA GGC TCC | 50 |
| H52A(−01+24) | CTG TTC CAA ATC CTG CAT TGT TGC C | 51 |

wherein A is 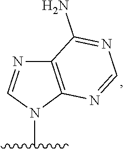 [SMILES: *n1cnc2c(N)ncnc21]
C is 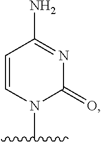 [SMILES: *n1ccc(N)nc1=O]
G is 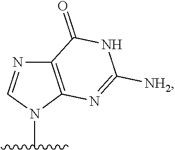 [SMILES: *n1cnc2c(=O)[nH]c(N)nc21]
T is 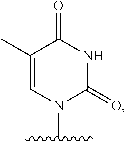 [SMILES: *n1cc(C)c(=O)[nH]c1=O]
U is 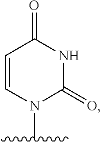 [SMILES: *n1ccc(=O)[nH]c1=O]
Gm is methylated guanine, Am is methylated adenine, and m5C is

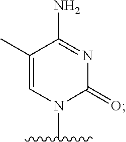 [SMILES: *n1cc(C)c(N)nc1=O]

30-35. (canceled)

36. The composition of claim 1, wherein the antisense oligonucleotide conjugate is of Formula (II): 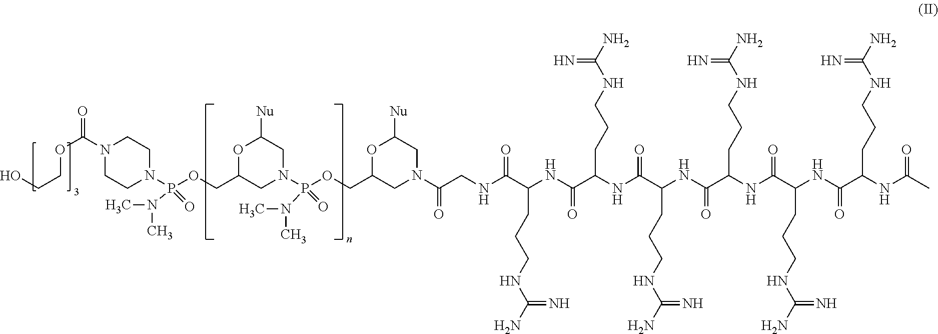
or a pharmaceutically acceptable salt thereof, each Nu from 1 to (n+1) and 5° to 3′ corresponds to the nucleobases in one of the following:

| | SEQ |
|---|---|
| | ID |
| Base Sequence [5′ to 3′] | NO. |
| CTC CAA CAT CAA GGA AGA TGG CAT TTC TAG | 1 |
| ACC TCC AAC ATC AAG GAA GAT GGC | 2 |
| GTA CCT CCA ACA TCA AGG AAG ATG GCA TTT | 3 |
| GTA CCT CCA ACA TCA AGG AAG ATG GCA T | 4 |
| TCA AGG AAG ATG GCA TTT CT | 5 |
| UCA AGG AmAGm AmUGm GmCA UUU CU | 6 |
| GTT GCC TCC GGT TCT GAA GGT GTT C | 7 |
| GTT G5mC5mC T5mC5mC GGT T5mC T GAA GGT GTT | 8 |
| 5mC | |
| CCT CCG GTT CTG AAG GTG TTC | 9 |
| CTG AAG GTG TTC TTG TAC TTC ATC C | 10 |
| CCT CCG GTT CTG AAG GTG TTC TTG T | 11 |
| GTT GCC TCC GGT TCT GAA GGT GTT CTT G | 12 |
| TTG CCT CCG GTT CTG AAG GTG TTC TTG TAC | 13 |
| CTG TTG CCT CCG GTT CTG AAG GTG | 14 |
| CAT TCA ACT GTT GCC TCC GGT TCT GAA GGT G | 15 |
| CTG TTG CCT CCG GTT CTG | 16 |
| CAA TGC CAT CCT GGA GTT CCT G | 17 |
| GCT GCC CAA TGC CAT CCT GGA GTT CCT GTA AGA | 18 |
| T | |
| GCT GCC CAA TGC CAT CCT GGA GTT CCT G | 19 |
| GCT GCC CAA TGC CAT CCT GGA GTT CCT GTA A | 20 |
| CAA TGC CAT CCT GGA GTT CCT GTA AGA TAC C | 21 |
| CAA TGC CAT CCT GGA GTT CCT GTA AGA T | 22 |
| TGC CAT CCT GGA GTT CCT GTA AGA TAC C | 23 |
| GCT GCC CAA TGC CAT CCT GGA GTT CCT GTA AGA | 24 |
| TAC CAA | |
| CAA TGC CAT CCT GGA GTT CCT GTA AGA | 25 |
| GCT GCC CAA TGC CAT CCT GGA GTT CCT GTA AG | 26 |
| GCC CAA TGC CAT CCT GGA GTT CCT GTA AGA TAC | 27 |
| C | |
| GCC CAA TGC CAT CCT GGA GTT CCT GTA AGA T | 28 |
| TTG CCG CTG CCC AAT GCC ATC CTG GAG TTC CTG | 29 |
| TAA GAT | |
| GCC CAA TGC CAT CCT GGA GTT CCT GTA A | 30 |
| GCC GCT GCC CAA TGC CAT CCT GGA GTT CCT GTA | 31 |
| A | |
| GCC CAA TGC CAT CCT GGA GTT CCT G | 32 |
| GCC GCT GCC CAA TGC CAT CCT GGA GTT CCT G | 33 |
| m5C-G-m5C-T-G-C-m5C-m5C-A-A-T-G-m5C-m5C-A-U- | 34 |
| m5C-m5C | |
| GAT CTG TCA AAT CGC CTG CAG GTA A | 35 |
| GAT CTG TCA AAT CGC CTG CAG G | 36 |
| CAG ATC TGT CAA ATC GCC TGC AGG | 37 |
| GAT CTG TCA AAT CGC CTG CAG GT | 38 |
| GAT CTG TCA AAT CGC CTG CAG | 39 |
| CAG ATC TGT CAA ATC GCC TGC AGG T | 40 |
| CAG ATC TGT CAA ATC GCC TGC AG | 41 |
| GGG ATC CAG TAT ACT TAC AGG C | 42 |
| GGG ATC CAG TAT ACT TAC AGG CTC C | 43 |
| GAT CCA GTA TAC TTA CAG GCT CC | 44 |
| GGA TCC AGT ATA CTT ACA GGC TCC | 45 |
| ACT TCC TCT TTA ACA GAA AAG CAT AC | 46 |
| ATC CAG TAT ACT TAC AGG CTC C | 47 |
| GAG CTC AGA TCT TCT AAC TTC CTC T | 48 |
| GGG ATC CAG TAT ACT TAC AGG CTC | 49 |
| ATG GGA TCC AGT ATA CTT ACA GGC TCC | 50 |
| CTG TTC CAA ATC CTG CAT TGT TGC C | 51 |

wherein A is 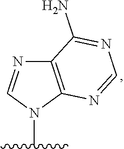 [SMILES: *n1cnc2c(N)ncnc21]
C is 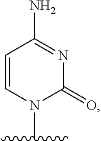 [SMILES: *n1ccc(N)nc1=O]
G is 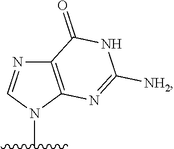 [SMILES: *n1cnc2c(=O)[nH]c(N)nc21]
T is 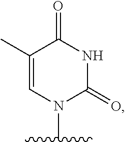 [SMILES: *n1cc(C)c(=O)[nH]c1=O]
U is 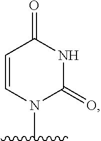 [SMILES: *n1ccc(=O)[nH]c1=O]
Gm is methylated guanine, Am is methylated adenine, and m5C is

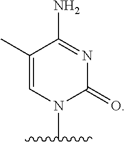 [SMILES: *n1cc(C)c(N)nc1=O]

37-41. (canceled)

42. The composition of claim 1, wherein the antisense oligonucleotide conjugate is of Formula (III): 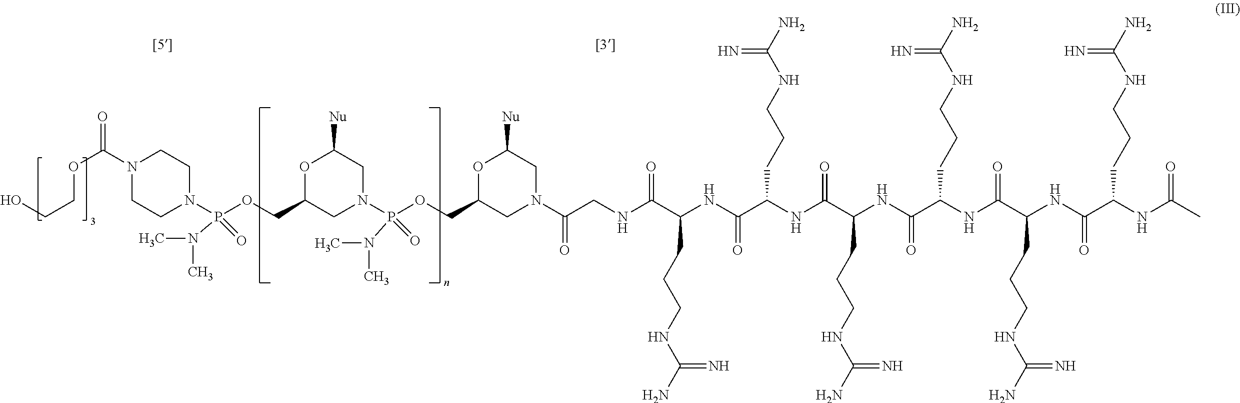
or a pharmaceutically acceptable salt thereof, each Nu from 1 to (n+1) and 5′ to 3′ corresponds to the nucleobases in one of the following:

| | SEQ |
|---|---|
| | ID |
| Base Sequence [5′ to 3′] | NO. |
| CTC CAA CAT CAA GGA AGA TGG CAT TTC TAG | 1 |
| ACC TCC AAC ATC AAG GAA GAT GGC | 2 |
| GTA CCT CCA ACA TCA AGG AAG ATG GCA TTT | 3 |
| GTA CCT CCA ACA TCA AGG AAG ATG GCA T | 4 |
| TCA AGG AAG ATG GCA TTT CT | 5 |
| UCA AGG AmAGm AmUGm GmCA UUU CU | 6 |
| GTT GCC TCC GGT TCT GAA GGT GTT C | 7 |
| GTT G5mC5mC T5mC5mC GGT T5mC T GAA GGT GTT | 8 |
| 5mC | |
| CCT CCG GTT CTG AAG GTG TTC | 9 |
| CTG AAG GTG TTC TTG TAC TTC ATC C | 10 |
| CCT CCG GTT CTG AAG GTG TTC TTG T | 11 |
| GTT GCC TCC GGT TCT GAA GGT GTT CTT G | 12 |
| TTG CCT CCG GTT CTG AAG GTG TTC TTG TAC | 13 |
| CTG TTG CCT CCG GTT CTG AAG GTG | 14 |
| CAT TCA ACT GTT GCC TCC GGT TCT GAA GGT G | 15 |
| CTG TTG CCT CCG GTT CTG | 16 |
| CAA TGC CAT CCT GGA GTT CCT G | 17 |
| GCT GCC CAA TGC CAT CCT GGA GTT CCT GTA AGA | 18 |
| T | |
| GCT GCC CAA TGC CAT CCT GGA GTT CCT G | 19 |
| GCT GCC CAA TGC CAT CCT GGA GTT CCT GTA A | 20 |
| CAA TGC CAT CCT GGA GTT CCT GTA AGA TAC C | 21 |
| CAA TGC CAT CCT GGA GTT CCT GTA AGA T | 22 |
| TGC CAT CCT GGA GTT CCT GTA AGA TAC C | 23 |
| GCT GCC CAA TGC CAT CCT GGA GTT CCT GTA AGA | 24 |
| TAC CAA | |
| CAA TGC CAT CCT GGA GTT CCT GTA AGA | 25 |
| GCT GCC CAA TGC CAT CCT GGA GTT CCT GTA AG | 26 |
| GCC CAA TGC CAT CCT GGA GTT CCT GTA AGA TAC | 27 |
| C | |
| GCC CAA TGC CAT CCT GGA GTT CCT GTA AGA T | 28 |
| TTG CCG CTG CCC AAT GCC ATC CTG GAG TTC CTG | 29 |
| TAA GAT | |
| GCC CAA TGC CAT CCT GGA GTT CCT GTA A | 30 |
| GCC GCT GCC CAA TGC CAT CCT GGA GTT CCT GTA A | 31 |
| GCC CAA TGC CAT CCT GGA GTT CCT G | 32 |
| GCC GCT GCC CAA TGC CAT CCT GGA GTT CCT G | 33 |
| m5C-G-m5C-T-G-C-m5C-m5C-A-A-T-G-m5C-m5C-A-U- | 34 |
| m5C-m5C | |
| GAT CTG TCA AAT CGC CTG CAG GTA A | 35 |
| GAT CTG TCA AAT CGC CTG CAG G | 36 |
| CAG ATC TGT CAA ATC GCC TGC AGG | 37 |
| GAT CTG TCA AAT CGC CTG CAG GT | 38 |
| GAT CTG TCA AAT CGC CTG CAG | 39 |
| CAG ATC TGT CAA ATC GCC TGC AGG T | 40 |
| CAG ATC TGT CAA ATC GCC TGC AG | 41 |
| GGG ATC CAG TAT ACT TAC AGG C | 42 |
| GGG ATC CAG TAT ACT TAC AGG CTC C | 43 |
| GAT CCA GTA TAC TTA CAG GCT CC | 44 |
| GGA TCC AGT ATA CTT ACA GGC TCC | 45 |
| ACT TCC TCT TTA ACA GAA AAG CAT AC | 46 |
| ATC CAG TAT ACT TAC AGG CTC C | 47 |
| GAG CTC AGA TCT TCT AAC TTC CTC T | 48 |
| GGG ATC CAG TAT ACT TAC AGG CTC | 49 |
| ATG GGA TCC AGT ATA CTT ACA GGC TCC | 50 |
| CTG TTC CAA ATC CTG CAT TGT TGC C | 51 |

wherein A is 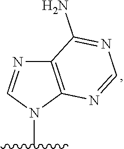 [SMILES: *n1cnc2c(N)ncnc21]
C is 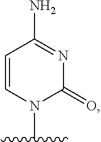 [SMILES: *n1ccc(N)nc1=O]
G is 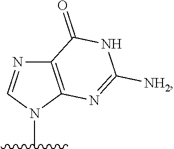 [SMILES: *n1cnc2c(=O)[nH]c(N)nc21]
T is 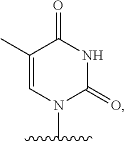 [SMILES: *n1cc(C)c(=O)[nH]c1=O]
U is 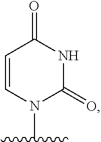 [SMILES: *n1ccc(=O)[nH]c1=O]
Gm is methylated guanine, Am is methylated adenine, and m5C is

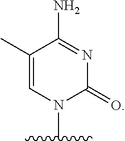 [SMILES: *n1cc(C)c(N)nc1=O]

43-47. (canceled)

48. The composition of claim 1, wherein the antisense oligonucleotide conjugate is PPMO #1, PPMO #2, PPMO #3, or PPMO #4 or a pharmaceutically acceptable salt thereof.

49. (canceled)

50. The composition of claim 1, wherein the antisense oligonucleotide conjugate is a pharmaceutically acceptable salt.

51-53. (canceled)

54. The composition of claim 50, wherein the antisense oligonucleotide conjugate is the 6HCl salt form of PPMO #1, PPMO #2, PPMO #3, or PPMO #4.

55-58. (canceled)

59. A method of treating a human patient having Duchenne muscular dystrophy, comprising dissolving or dispersing the composition of claim 1 in an aqueous solvent.

60. The method of claim 59, wherein the amount of aqueous solvent used to dissolve or disperse the composition is sufficient to provide a concentration of the antisense oligonucleotide conjugate of about 20 mg/mL to about 200 mg/mL.

61-63. (canceled)


SEQUENCE LISTING (60 sequences)

SEQ ID NO: 1 - DNA, 30 bp, Artificial Sequence
  source: /note=“Description of Artificial Sequence: Synthetic oligonucleotide”
ctccaacatcaaggaagatggcatttctag

SEQ ID NO: 2 - DNA, 24 bp, Artificial Sequence
  source: /note=“Description of Artificial Sequence: Synthetic oligonucleotide”
acctccaacatcaaggaagatggc

SEQ ID NO: 3 - DNA, 30 bp, Artificial Sequence
  source: /note=“Description of Artificial Sequence: Synthetic oligonucleotide”
gtacctccaacatcaaggaagatggcattt

SEQ ID NO: 4 - DNA, 28 bp, Artificial Sequence
  source: /note=“Description of Artificial Sequence: Synthetic oligonucleotide”
gtacctccaacatcaaggaagatggcat

SEQ ID NO: 5 - DNA, 20 bp, Artificial Sequence
  source: /note=“Description of Artificial Sequence: Synthetic oligonucleotide”
tcaaggaagatggcatttct

SEQ ID NO: 6 - RNA, 20 nt, Artificial Sequence
  source: /note=“Description of Artificial Sequence: Synthetic oligonucleotide”
ucaaggaagauggcauuucu

SEQ ID NO: 7 - DNA, 25 bp, Artificial Sequence
  source: /note=“Description of Artificial Sequence: Synthetic oligonucleotide”
gttgcctccggttctgaaggtgttc

SEQ ID NO: 8 - DNA, 25 bp, Artificial Sequence
  source: /note=“Description of Artificial Sequence: Synthetic oligonucleotide”
gttgcctccggttctgaaggtgttc

SEQ ID NO: 9 - DNA, 21 bp, Artificial Sequence
  source: /note=“Description of Artificial Sequence: Synthetic oligonucleotide”
cctccggttctgaaggtgttc

SEQ ID NO: 10 - DNA, 25 bp, Artificial Sequence
  source: /note=“Description of Artificial Sequence: Synthetic oligonucleotide”
ctgaaggtgttcttgtacttcatcc

SEQ ID NO: 11 - DNA, 25 bp, Artificial Sequence
  source: /note=“Description of Artificial Sequence: Synthetic oligonucleotide”
cctccggttctgaaggtgttcttgt

SEQ ID NO: 12 - DNA, 28 bp, Artificial Sequence
  source: /note=“Description of Artificial Sequence: Synthetic oligonucleotide”
gttgcctccggttctgaaggtgttcttg

SEQ ID NO: 13 - DNA, 30 bp, Artificial Sequence
  source: /note=“Description of Artificial Sequence: Synthetic oligonucleotide”
ttgcctccggttctgaaggtgttcttgtac

SEQ ID NO: 14 - DNA, 24 bp, Artificial Sequence
  source: /note=“Description of Artificial Sequence: Synthetic oligonucleotide”
ctgttgcctccggttctgaaggtg

SEQ ID NO: 15 - DNA, 31 bp, Artificial Sequence
  source: /note=“Description of Artificial Sequence: Synthetic oligonucleotide”
cattcaactgttgcctccggttctgaaggtg

SEQ ID NO: 16 - DNA, 18 bp, Artificial Sequence
  source: /note=“Description of Artificial Sequence: Synthetic oligonucleotide”
ctgttgcctccggttctg

SEQ ID NO: 17 - DNA, 22 bp, Artificial Sequence
  source: /note=“Description of Artificial Sequence: Synthetic oligonucleotide”
caatgccatcctggagttcctg

SEQ ID NO: 18 - DNA, 34 bp, Artificial Sequence
  source: /note=“Description of Artificial Sequence: Synthetic oligonucleotide”
gctgcccaatgccatcctggagttcctgtaagat

SEQ ID NO: 19 - DNA, 28 bp, Artificial Sequence
  source: /note=“Description of Artificial Sequence: Synthetic oligonucleotide”
gctgcccaatgccatcctggagttcctg

SEQ ID NO: 20 - DNA, 31 bp, Artificial Sequence
  source: /note=“Description of Artificial Sequence: Synthetic oligonucleotide”
gctgcccaatgccatcctggagttcctgtaa

SEQ ID NO: 21 - DNA, 31 bp, Artificial Sequence
  source: /note=“Description of Artificial Sequence: Synthetic oligonucleotide”
caatgccatcctggagttcctgtaagatacc

SEQ ID NO: 22 - DNA, 28 bp, Artificial Sequence
  source: /note=“Description of Artificial Sequence: Synthetic oligonucleotide”
caatgccatcctggagttcctgtaagat

SEQ ID NO: 23 - DNA, 28 bp, Artificial Sequence
  source: /note=“Description of Artificial Sequence: Synthetic oligonucleotide”
tgccatcctggagttcctgtaagatacc

SEQ ID NO: 24 - DNA, 39 bp, Artificial Sequence
  source: /note=“Description of Artificial Sequence: Synthetic oligonucleotide”
gctgcccaatgccatcctggagttcctgtaagataccaa

SEQ ID NO: 25 - DNA, 27 bp, Artificial Sequence
  source: /note=“Description of Artificial Sequence: Synthetic oligonucleotide”
caatgccatcctggagttcctgtaaga

SEQ ID NO: 26 - DNA, 32 bp, Artificial Sequence
  source: /note=“Description of Artificial Sequence: Synthetic oligonucleotide”
gctgcccaatgccatcctggagttcctgtaag

SEQ ID NO: 27 - DNA, 34 bp, Artificial Sequence
  source: /note=“Description of Artificial Sequence: Synthetic oligonucleotide”
gcccaatgccatcctggagttcctgtaagatacc

SEQ ID NO: 28 - DNA, 31 bp, Artificial Sequence
  source: /note=“Description of Artificial Sequence: Synthetic oligonucleotide”
gcccaatgccatcctggagttcctgtaagat

SEQ ID NO: 29 - DNA, 39 bp, Artificial Sequence
  source: /note=“Description of Artificial Sequence: Synthetic oligonucleotide”
ttgccgctgcccaatgccatcctggagttcctgtaagat

SEQ ID NO: 30 - DNA, 28 bp, Artificial Sequence
  source: /note=“Description of Artificial Sequence: Synthetic oligonucleotide”
gcccaatgccatcctggagttcctgtaa

SEQ ID NO: 31 - DNA, 34 bp, Artificial Sequence
  source: /note=“Description of Artificial Sequence: Synthetic oligonucleotide”
gccgctgcccaatgccatcctggagttcctgtaa

SEQ ID NO: 32 - DNA, 25 bp, Artificial Sequence
  source: /note=“Description of Artificial Sequence: Synthetic oligonucleotide”
gcccaatgccatcctggagttcctg

SEQ ID NO: 33 - DNA, 31 bp, Artificial Sequence
  source: /note=“Description of Artificial Sequence: Synthetic oligonucleotide”
gccgctgcccaatgccatcctggagttcctg

SEQ ID NO: 34 - DNA, 18 bp, Artificial Sequence
  source: /note=“Description of Artificial Sequence: Synthetic oligonucleotide”
  source: /note=“Description of Combined DNA/RNA Molecule: Synthetic oligonucleotide”
cgctgcccaatgccaucc

SEQ ID NO: 35 - DNA, 25 bp, Artificial Sequence
  source: /note=“Description of Artificial Sequence: Synthetic oligonucleotide”
gatctgtcaaatcgcctgcaggtaa

SEQ ID NO: 36 - DNA, 22 bp, Artificial Sequence
  source: /note=“Description of Artificial Sequence: Synthetic oligonucleotide”
gatctgtcaaatcgcctgcagg

SEQ ID NO: 37 - DNA, 24 bp, Artificial Sequence
  source: /note=“Description of Artificial Sequence: Synthetic oligonucleotide”
cagatctgtcaaatcgcctgcagg

SEQ ID NO: 38 - DNA, 23 bp, Artificial Sequence
  source: /note=“Description of Artificial Sequence: Synthetic oligonucleotide”
gatctgtcaaatcgcctgcaggt

SEQ ID NO: 39 - DNA, 21 bp, Artificial Sequence
  source: /note=“Description of Artificial Sequence: Synthetic oligonucleotide”
gatctgtcaaatcgcctgcag

SEQ ID NO: 40 - DNA, 25 bp, Artificial Sequence
  source: /note=“Description of Artificial Sequence: Synthetic oligonucleotide”
cagatctgtcaaatcgcctgcaggt

SEQ ID NO: 41 - DNA, 23 bp, Artificial Sequence
  source: /note=“Description of Artificial Sequence: Synthetic oligonucleotide”
cagatctgtcaaatcgcctgcag

SEQ ID NO: 42 - DNA, 22 bp, Artificial Sequence
  source: /note=“Description of Artificial Sequence: Synthetic oligonucleotide”
gggatccagtatacttacaggc

SEQ ID NO: 43 - DNA, 25 bp, Artificial Sequence
  source: /note=“Description of Artificial Sequence: Synthetic oligonucleotide”
gggatccagtatacttacaggctcc

SEQ ID NO: 44 - DNA, 23 bp, Artificial Sequence
  source: /note=“Description of Artificial Sequence: Synthetic oligonucleotide”
gatccagtatacttacaggctcc

SEQ ID NO: 45 - DNA, 24 bp, Artificial Sequence
  source: /note=“Description of Artificial Sequence: Synthetic oligonucleotide”
ggatccagtatacttacaggctcc

SEQ ID NO: 46 - DNA, 26 bp, Artificial Sequence
  source: /note=“Description of Artificial Sequence: Synthetic oligonucleotide”
acttcctctttaacagaaaagcatac

SEQ ID NO: 47 - DNA, 22 bp, Artificial Sequence
  source: /note=“Description of Artificial Sequence: Synthetic oligonucleotide”
atccagtatacttacaggctcc

SEQ ID NO: 48 - DNA, 25 bp, Artificial Sequence
  source: /note=“Description of Artificial Sequence: Synthetic oligonucleotide”
gagctcagatcttctaacttcctct

SEQ ID NO: 49 - DNA, 24 bp, Artificial Sequence
  source: /note=“Description of Artificial Sequence: Synthetic oligonucleotide”
gggatccagtatacttacaggctc

SEQ ID NO: 50 - DNA, 27 bp, Artificial Sequence
  source: /note=“Description of Artificial Sequence: Synthetic oligonucleotide”
atgggatccagtatacttacaggctcc

SEQ ID NO: 51 - DNA, 25 bp, Artificial Sequence
  source: /note=“Description of Artificial Sequence: Synthetic oligonucleotide”
ctgttccaaatcctgcattgttgcc

SEQ ID NO: 52 - protein, 7 aa, Artificial Sequence
  source: /note=“Description of Artificial Sequence: Synthetic peptide”
RRRRRRG

SEQ ID NO: 53 - protein, 6 aa, Artificial Sequence
  source: /note=“Description of Artificial Sequence: Synthetic peptide”
RRRRRR

SEQ ID NO: 54 - protein, 12 aa, Artificial Sequence
  source: /note=“Description of Artificial Sequence: Synthetic peptide”
  MOD_RES (2)..(2): 6-aminohexanoic acid
  MOD_RES (5)..(5): 6-aminohexanoic acid
  MOD_RES (8)..(8): 6-aminohexanoic acid
  MOD_RES (11)..(11): 6-aminohexanoic acid
RXRRXRRXRRXR

SEQ ID NO: 55 - protein, 10 aa, Artificial Sequence
  source: /note=“Description of Artificial Sequence: Synthetic peptide”
RFFRFFRFFR

SEQ ID NO: 56 - protein, 14 aa, Artificial Sequence
  source: /note=“Description of Artificial Sequence: Synthetic peptide”
  MOD_RES (2)..(2): 6-aminohexanoic acid
  MOD_RES (5)..(5): 6-aminohexanoic acid
  MOD_RES (8)..(8): 6-aminohexanoic acid
  MOD_RES (11)..(11): 6-aminohexanoic acid
  MOD_RES (13)..(13): 6-aminohexanoic acid
  MOD_RES (14)..(14): Beta-alanine
RXRRXRRXRRXRXX

SEQ ID NO: 57 - protein, 12 aa, Artificial Sequence
  source: /note=“Description of Artificial Sequence: Synthetic peptide”
  MOD_RES (11)..(11): 6-aminohexanoic acid
  MOD_RES (12)..(12): Beta-alanine
RFFRFFRFFRXX

SEQ ID NO: 58 - protein, 11 aa, Artificial Sequence
  source: /note=“Description of Artificial Sequence: Synthetic peptide”
RFFRFFRFFRG

SEQ ID NO: 59 - protein, 6 aa, Artificial Sequence
  source: /note=“Description of Artificial Sequence: Synthetic peptide”
RRRRRG

SEQ ID NO: 60 - protein, 5 aa, Artificial Sequence
  source: /note=“Description of Artificial Sequence: Synthetic peptide”
RRRRR